(19) United States
(12) Patent Application Publication
(10) Pub. No.: US 2019/0019603 A1
(43) Pub. Date: Jan. 17, 2019

(54) PRINTED RESISTORS FOR BIOPOTENTIAL SENSOR SYSTEMS AND METHODS

(71) Applicant: General Electric Company, Schenectady, NY (US)

(72) Inventors: Azar Alizadeh, Schenectady, NY (US); Matthew Jeremiah Misner, Delanson, NY (US); Andrew A. Burns, Schenectady, NY (US); Juha Virtanen, Helsinki (FI); Otto Pekander, Helsinki (FI); Robert Santala, Helsinki (FI); Teemu Alajoki, Oulu (FI)

(21) Appl. No.: 16/026,955

(22) Filed: Jul. 3, 2018

Related U.S. Application Data
(60) Provisional application No. 62/531,099, filed on Jul. 11, 2017.

(51) Int. Cl. H01C 1/16; H01C 7/00; H01C 1/14; H01C 17/065; H01C 1/08; H05K 1/16; A61B 5/0424; A61B 5/0408

(52) U.S. Cl. CPC H01C 1/16; H01C 7/003; H01C 1/14; H01C 17/065; H01C 1/08; H05K 1/167; A61N 1/39; A61B 5/04085; H05K 2201/10151; A61B 2562/0209; A61B 2562/125; A61B 2562/164; A61B 2562/166; A61B 5/0424

(57) ABSTRACT

A system includes a conformable biopotential sensor that withstands a defibrillation pulse. The conformable biopotential sensor includes a polymer substrate, a plurality of electrodes printed on the polymer substrate, a signal trace printed on the polymer substrate, and one or more resistors printed on the polymer substrate and in electrical communication with an electrode of the plurality of electrodes via the signal trace. One or both of the one or more resistors and the polymer substrate withstand a defibrillation pulse. The conformable biopotential sensor further includes a coating layer applied to the one of both of the one or more resistors, wherein the coating is more thermally conductive than the polymer substrate.

### CROSS-REFERENCE TO RELATED APPLICATIONS

This application claims priority to U.S. Patent Provisional Application No. 62/531,099, entitled “PRINTER LEAD-SET WITH DEFIBRILLATION PROTECTION RESISTORS FOR ECG”, filed Jul. 11, 2017, which is herein incorporated by reference in its entirety.

### BACKGROUND

The subject matter disclosed herein relates to biopotential sensor systems and methods. In particular, the subject matter disclosed herein relates to systems and methods for at least one defibrillation protection resistor for use with bioparameter sensor applications.

Clinicians are interested in monitoring various physiological parameters of a patient. In some instances, the parameters associated with the heart may provide information about a patient's health or condition. For example, such parameters may include heart rate, heart rhythm, etc. Certain monitoring devices, such as electrocardiography (ECG) devices, may involve recording electrical activity associated with the heart over a period of time using any number of electrodes in contact with the skin. For instance, a conventional twelve-lead ECG device may include electrodes placed on the limbs of a patient and on the surface of the chest. The measured overall magnitude of the heart's electrical potential may be measured and recorded over a period of time to capture the overall magnitude and the direction of the heart's electrical depolarization at each moment through the cardiac cycle.

In certain ECG devices, there are high voltage resistors in series with each electrode lead wiring (e.g., signal trace) to prevent excessive current flowing through the electrodes when the electrodes are used to monitor cardiac activity during the application of defibrillation shocks. During defibrillation, a defibrillator delivers a dose of electric current to the heart to attempt to re-establish normal sinus rhythm of the heart. Resistors used in conjunction with defibrillators that meet the pulse energy tolerance associated with defibrillation shocks are typically large and require extensive manufacturing, rendering these resistors expensive and too large for certain applications. As such, it may be beneficial to develop smaller and more economic systems and methods that provide protection from signal pulses, such as defibrillation pulses in ECG devices.

### BRIEF DESCRIPTION

In one embodiment, a system includes a conformable biopotential sensor that withstands a defibrillation pulse. The conformable biopotential sensor includes a polymer substrate, a plurality of electrodes printed on the polymer substrate, a signal trace printed on the polymer substrate, and one or more resistors printed on the polymer substrate and in electrical communication with an electrode of the plurality of electrodes via the signal trace. One or both of the one or more resistors and the polymer substrate withstand a defibrillation pulse. The conformable biopotential sensor further includes a coating layer applied to the one of both of the one or more resistors, such that the coating is more thermally conductive than the polymer substrate.

In another embodiment, a method of manufacturing a biopotential sensor includes printing a plurality of electrodes onto a polymer substrate, such that the polymer substrate is conformable, printing one or more resistors onto the polymer substrate, printing a signal trace onto the polymer substrate, such that the signal trace electrically couples the electrode and the one or more resistors, and applying a coating material more thermally conductive than the polymer substrate to the one or more resistors, such that the coating material dissipates heat of the one or more resistors during operation of the biopotential sensor.

In yet another embodiment, a system comprises a biopotential sensor, which includes a plurality of printed electrodes, such that the plurality of printed electrodes contact a user to detect a bioparameter associated with the user. Furthermore, the biopotential sensor includes one or more printed resistors in electrical communication with an electrode of the plurality of electrodes via a printed signal trace, such that the plurality of printed electrodes, the one or more printed resistors, and the printed signal trace are printed on a polymer substrate. The biopotential sensor also includes a coating applied to the one or more resistors, wherein the coating is more thermally conductive than the polymer substrate.

### BRIEF DESCRIPTION OF THE DRAWINGS

These and other features, aspects, and advantages of the present disclosure will become better understood when the following detailed description is read with reference to the accompanying drawings in which like characters represent like parts throughout the drawings, wherein:

FIG. 1 is a schematic diagram of an embodiment of a defibrillator configured for use in conjunction with a biopotential sensor, such as an ECG device, in accordance with aspects of the present disclosure;

FIG. 2 is a schematic diagram of an embodiment of a variety of printed resistors with different geometries for use in the biopotential sensor of FIG. 1, in accordance with aspects of the present disclosure;

FIG. 3A is a schematic diagram of an embodiment of a thermal image of a meandered resistor of the variety of resistors of FIG. 2, in accordance with aspects of the present disclosure;

FIG. 3B is a schematic diagram of an embodiment of a three-dimensional (3D) meandered resistor of the variety of resistors of FIG. 2, in accordance with aspects of the present disclosure;

FIG. 3C is a schematic diagram of an embodiment of a 3D meandered resistor of the variety of resistors of FIG. 2, in accordance with aspects of the present disclosure;

FIG. 3D is a schematic diagram of an embodiment of 3D meandered resistor of the variety of resistors of FIG. 2, in accordance with aspects of the present disclosure;

FIG. 4A is a schematic diagram of an embodiment of a printed ECG, in accordance with aspects of the present disclosure;

FIG. 4B shows arrangements of the printed resistors of FIG. 2, in accordance with aspects of the present disclosure;

FIG. 5 is a schematic diagram of an embodiment of the printed ECG of FIG. 4A printed onto a Thermoplastic Polyurethane (TPU) substrate, in accordance with aspects of the present disclosure;

FIG. 6 is a schematic diagram of another embodiment of the printed ECG of FIG. 4A printed onto a foldable TPU substrate, in accordance with aspects of the present disclosure;

FIG. 7 is a schematic diagram of silver wiring printed onto the foldable TPU substrate of FIG. 6, in accordance with aspects of the present disclosure;

FIG. 8 is a schematic diagram of another embodiment of the variety of printed resistors of FIG. 2 included in the ECG of FIG. 4A printed onto the foldable TPU substrate of FIG. 6, in accordance with aspects of the present disclosure; and

FIG. 9 is a line graph of the resistance for various resistors corresponding to various test points, in accordance with aspects of the present disclosure;

FIG. 10 is another line graph of the resistor deviation for various resistors corresponding to various test points, in accordance with aspects of the present disclosure;

FIG. 11 is another line graph of the resistance for various resistor placements, resistor types, and inks corresponding to various test patterns, in accordance with aspects of the present disclosure;

FIG. 12 is a schematic diagram of an embodiment of a vertical meandered resistor and a horizontal meandered resistor of the variety of printed resistors of FIG. 2, in accordance with aspects of the present disclosure;

FIG. 13 is another line graph of the resistance against the resistor orientation and resistor layer configuration for a Henkel Resistor, in accordance with aspects of the present disclosure;

FIG. 14 is another line graph of the resistance against the resistor orientation and resistor layer configuration for the PPG XCMC-50 ink, in accordance with aspects of the present disclosure;

FIG. 15 is a schematic diagram of an embodiment of a printed respiratory prototype, in accordance with aspects of the present disclosure;

FIG. 16 is a schematic diagram of an embodiment of a portion of the printed respiratory prototype of FIG. 15 and testing measurement locations, in accordance with aspects of the present disclosure;

FIG. 17 is a schematic diagram of an embodiment of printed resistors employed in the printed respiratory prototype of FIG. 15, in accordance with aspects of the present disclosure;

FIG. 18 is a schematic diagram of an embodiment of a printer configured to print the printed resistors of FIG. 17, in accordance with aspects of the present disclosure;

FIG. 19 is a schematic diagram of an embodiment of the printed resistors of FIG. 17, in accordance with aspects of the present disclosure;

FIG. 20 is a line graph of the measured resistance value of meandered resistors having various resistance values corresponding to various resistor inks, in accordance with aspects of the present disclosure;

FIG. 21 is a line graph of the measured resistance value of straight resistors having various resistance values corresponding to various resistor inks, in accordance with aspects of the present disclosure; and

FIG. 22 is a line graph of the measured resistance value of the Henkel resistors of FIG. 13 having various resistance values corresponding to various resistor inks, in accordance with aspects of the present disclosure.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present disclosure, the articles “a,” “an,” and “the” are intended to mean that there are one or more of the elements. The terms “comprising,” “including,” and “having” are intended to be inclusive and mean that there may be additional elements other than the listed elements.

While the following discussion is generally provided in the context of a printed lead-set that includes resistors that provide protection against exposure to high energy, for example, during defibrillation in medical applications, such as ECG devices, it should be appreciated that the embodiments disclosed herein are not limited to such medical contexts. Indeed, the provision of examples and explanations in such a medical context is only to facilitate explanation by providing instances of real-world implementations and applications. For example, the embodiments disclosed herein may be useful in a blood pressure sensors, pulse oximeter sensors, electromyography (EMG) sensors, inertial sensors, respiratory sensors, and other suitable biopotential sensors. The embodiments of the present disclosure may also be implemented to print resistors in any suitable electrical application, such as consumer electronics, television sets, lighting systems, and so forth.

In particular, the present embodiments address certain issues arising when a device experiences a pulse signal (e.g., voltage spike), such as during defibrillation. For example, in typical ECG devices, there are one or more high voltage resistors in series with each electrode lead wire(s) (e.g., signal trace(s)) to prevent excessive current flowing through the electrodes in case of defibrillation. During defibrillation, a defibrillator delivers a dose of electric current to the heart to attempt to re-establish normal sinus rhythm of the heart, such that the ECG device does not draw more than a certain percentage (e.g., ten percent) of the total energy from a defibrillation pulse with a peak amplitude of about 5 kV.

Although certain implementations of biopotential sensors may include resistors capable of withstanding defibrillation pulses, challenges exist in reducing the footprint and cost of such resistors. For example, while printed biopotential sensors including printed electrodes and leads have manufacturing advantages, such printing methods may introduce structural elements that decrease the ability of the biopotential sensors to withstand defibrillation pulses. For example, linear resistor geometries, while larger, provide more robust protection against breakage caused by defibrillation pulses. While printing methods permit implementing resistors with turns or other geometries associated with smaller footprints, such structures may increase the potential for breakage of the sensor. For example, the current of a defibrillation pulse may pack closer to the inner edges of turns in resistors, which results in concentration points for defects and an increased likelihood of resistor breakage.

Further while printing methods may provide manufacturing advantages, such methods have not been used in biopotential sensors because of the decreased heat tolerance (and increased breaking potential) associated with such manufacturing applications. The printed resistors and/or lead sets provided herein have improved heat tolerance. Provided herein are resistors that have improved energy dissipation characteristics that permit linear and nonlinear resistor geometries and, in turn, may be implemented with the overall smaller footprints associated with nonlinear geometries in some embodiments. In one embodiment, regardless of the resistor geometry the resistors are printed on substrates (e.g., polymer substrates) with improved heat dissipation characteristics to improve robustness. In another embodiment, the ink used to print the resistor may have resistance to cracking. Further, the heat dissipating substrate and crack-resistant ink may be used alone or in combination with one another and with or without smaller footprint resistor geometries. In yet another embodiment, the biopotential sensors may include one or more coating elements that have larger heat capacity than the substrate and/or are more thermally conductive than the substrate.

With the foregoing in mind, provided herein are printed biopotential sensors configured to withstand a pulse signal, for example, during defibrillation. The printed biopotential sensors may provide advantages of lower component cost and simplified device construction. The printed biopotential sensors may be printed according to the embodiments disclosed herein. In some embodiments, the biopotential sensors may be printed using roll-to-roll methods, such that various layers may be printed onto a flexible substrate. In some embodiments, the substrate may be of any suitable material that folds and/or stretches, such as thermoplastic polyurethane (TPU).

In some embodiments, a variety of electrodes, a variety of resistors, and a variety of signal traces (e.g., wiring) may be printed onto the substrate to be used as a biopotential sensor. For example, the electrodes may be printed with an ink having a composition that includes a silver/silver-chloride (Ag/AgCl) solution. The signal traces may be printed with an ink having Ag. Further, the printed resistors may be printed in various sizes (e.g., length, width, shape, etc.) with various inks, as described in detail below. In some embodiments, the resistors may be coated with a material thermally more conductive than that of the signal traces. In some embodiments, the shape and resistance value of the printed resistor and its coating may withstand defibrillation pulses and dissipate heat without delaminating from the rest of the components printed on the biopotential sensor. As provided herein, the printed biopotential sensor may be protected from defibrillation pulses, withstand high heat, include adhesive material to facilitate attachment to a user, require less resources to manufacture compared to conventional biopotential sensors, and be foldable to accommodate various users of various body types.

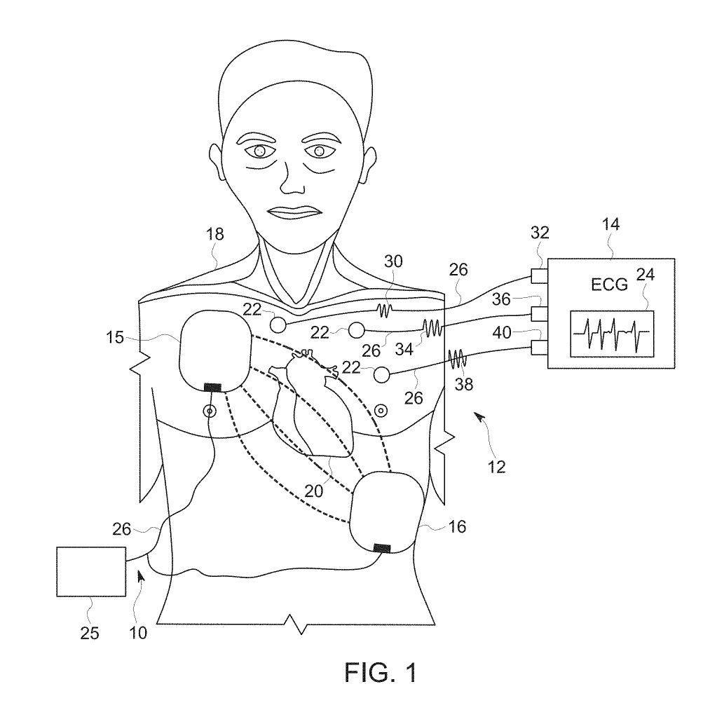

By way of introduction, FIG. 1 illustrates a schematic diagram of an embodiment of a defibrillation system 10 configured for use in conjunction with a biopotential sensor 12 coupled to a medical monitor 14, such as an ECG device. In the illustrated embodiment, the defibrillation system 10 includes a first defibrillation electrode 15 and a defibrillation electrode 16 applied to a patient 18 (e.g., person). While the depicted biopotential sensors 12 are shown in conjunction with the defibrillation system 10 by way of example, it should be understood that the defibrillation resistant biopotential sensors 12 may be used in other contexts for cardiac or biopotential monitoring and not solely in conjunction with defibrillation systems 10.

In some embodiments, the defibrillation system 10 may be used to treat certain medical conditions, such as cardiac dysrhythmias, for example, by delivering a dose of electric current to the heart 20. In the illustrated embodiment, the medical monitor 14 includes multiple electrodes 22 that record the electrical activity of the heart 20. Specifically, the electrodes 22 may detect electrical changes on the skin that arise from the heart's muscle's electrophysiological pattern of depolarizing and repolarizing during a heartbeat. In some instances, the medical monitor 14 may include a display 24 to present a graph of the electrical activity of the heart over time.

In some embodiments, the electrodes 15, 16 used to deliver the electric current to the heart may include certain features present in the biopotential sensor 12 as provided herein. Further, it should be noted that the defibrillation system 10 may include any suitable number of defibrillation electrodes and biopotential sensor electrodes 22. Further, the defibrillation system 10, including a pulse generator 25 to provide defibrillation pulses, and the medical monitor 12 may be incorporated into one device. In some embodiments, the biopotential sensor 12 includes the electrodes (e.g., electrodes 22) coupled the lead wires 26 (e.g., signal traces), which may include any suitable number of resistors. In particular, the electrodes 22, lead wires 26 and the resistors may be printed as described in detail below.

It should be noted that the dimensions of the printed resistor may depend on the energy the printed resistor draws from the defibrillation pulse. For example, for printed resistors having a higher resistance, the amount of absorbed energy may be less. In some instances, there may be up to three resistors or more resistors connected to one wiring (e.g., having a signal trace). For example, in the illustrated embodiment, a first resistor 30 having a resistance of about 100 kilo ohms (kΩ) may be associated with the ECG amplifier input 32. Further, a second resistor 34 having a resistance of about 25 kΩ may be associated with a respiration excitation signal output 36. Even further, a third resistor 38 having a resistance of about 50 kΩ may be associated with a respiration amplifier 40. It should be understood that in some embodiments, any of the aforementioned resistors (e.g., the third resistor) may be omitted. For example, when the third resistor 38 is omitted, the respiration signal from the respiration amplifier 40 may be read via the first resistor.

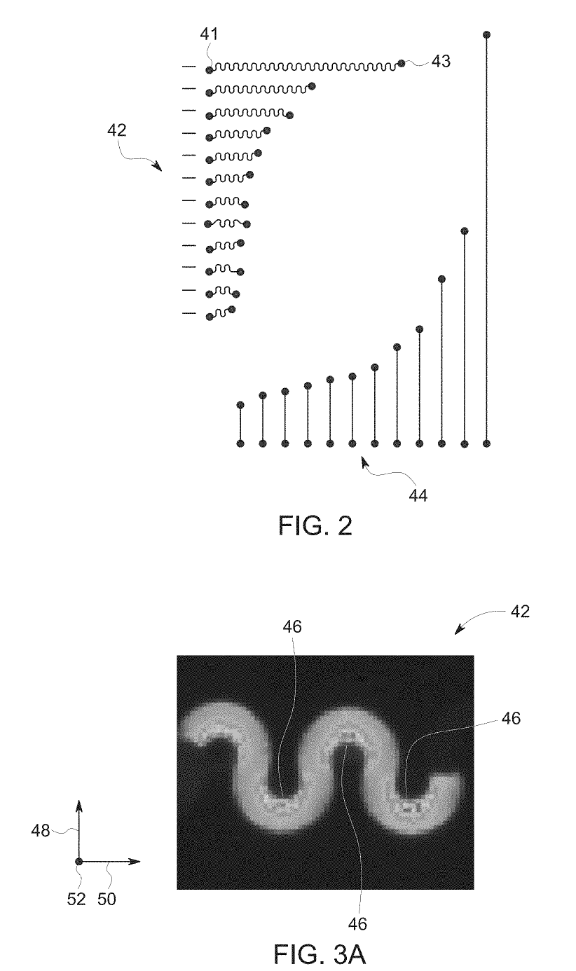

FIG. 2 is a schematic diagram of an embodiment of a variety of printed resistors with different geometries for use in the biopotential sensor of FIG. 1, in accordance with aspects of the present disclosure. In particular, the printed resistors may be nonlinear (e.g., meandering or curved) resistors 42, straight resistors 44, or a combination thereof. There are different aspects to consider when choosing the shape of the printed resistor. For example, with typical printing processes, the width of a resistor trace may be about 2 mm and the length may be about 80 mm. The minimum width is determined by the tolerances in the printing process, concerns for the absolute values, and considerations for matching of the resistances. The total length of the resistor can be reduced my meandering the shape of the resistor, as depicted in the illustrate embodiment. Further, in the illustrated embodiment, the straight resistors 44 and the corresponding nonlinear resistors 42 may be of a similar length. It should be noted that the resisters may be printed using other printing methods, such as top-down methods (e.g., lithography, chemical vapor deposition, etc.) and other printing methods.

As shown, the nonlinear resistors 42 may include any suitable geometries and may, in certain embodiments, include a mixture of straight and nonlinear sections. The nonlinear geometries may include one or more hairpin turns, angles, rounded ends, reversals, etc., such that a total length of the nonlinear resistor 42 between a first end 41 and a second end 43 is longer than a straight line drawn between the first end 41 and the second end 43. The first end 41 and the second end 43 may represent electrical contact points to the signal trace as provided herein.

In some embodiments, the resistors (e.g., the straight resistors 44 and the nonlinear resistors 42) may be printed on a polymer substrate. In some instances, the defibrillation pulse tolerances of the resistors printed on the polymer substrate depend primarily on the heat capacity of the printed resistor because the heat conduction to the polymer substrate may be too slow to take place during the duration of the defibrillation pulse (e.g., a few milliseconds). Accordingly, as provided herein, the power handling capability of the disclosed biopotential sensors may be improved by using substrates with better thermal conductivity and heat capacity. In this manner, the pulse tolerance of the printed resistor is improved by improved heat conduction to the substrate during the defibrillation pulse. Accordingly, the material of the substrate may be selected for improved thermal conductivity. Additionally, the localized heating effects during defibrillation pulses may be reduced by distributing the energy dissipated in the electrodes into a phase-change material (e.g., Honeywell LTM6300-SP) with an appropriately chosen transition temperature and sufficient latent heat to lower the temperature effects on the ink and substrate. In some instances, the temperature effects may lead to thermal expansion coefficient mismatch, thermal ablation, or any other effects.

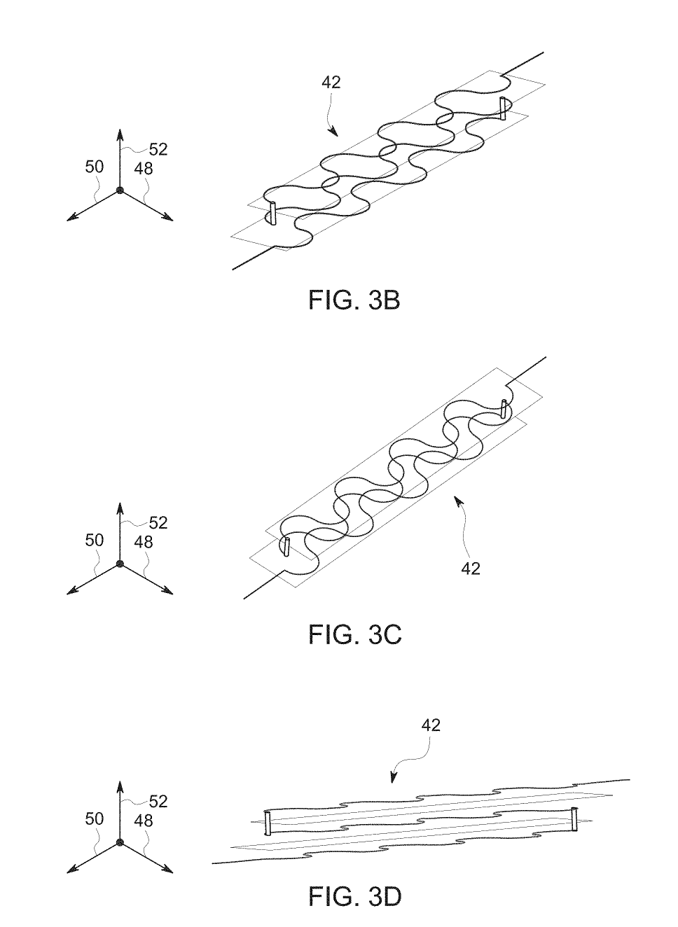

FIG. 3A is a schematic diagram of an embodiment of a thermal image of a nonlinear resistor 42 of the variety of resistors of FIG. 2, in accordance with aspects of the present disclosure. For nonlinear resistors 42, the current density may be higher close the inner corners 46, as depicted in the thermal image of the illustrated embodiment. For resistors with sharp corners, the current density may be higher near the edges. Accordingly, it may be beneficial to take into account this current density when implementing the nonlinear resistor 42, the straight resistors 44 (FIG. 2), or resistors of various shapes. For example, in some embodiments, the rounded edges of the nonlinear resistor 42 may be beneficial compared to sharp edges in this respect.

Furthermore, if a set of resistors are similar in length, the voltage distribution in the resistors may be similar, such that the resistors may be printed near one another. In some embodiments, a distance of about 4 mm between resistors is a suitable creepage distance for a 5 kV defibrillation pulse. It should be noted that the creepage distance may vary as the amplitude of the defibrillation pulse increases or decreases. In certain embodiments with more resistors, for example, when three resistors are employed, the distance between the three resistors may be less than 4 mm. In addition to meandering the printed resistors in a plane spanned by a first direction 48 and a second direction 50, in some embodiments, the printed resistors may also be nonlinear in a third direction 52 orthogonal to the plane spanned by the first and second direction 48, 50.

To help illustrate, FIGS. 3B-3D include three-dimensional (3D) printed resistors having one or more turns, curves, or angles, oriental along any of the first, second, and/or third directions 48, 50, 52. Using the third direction 52 for meandering the resistor provides benefits associated with an increase resistor area and a different geometry. For example, the printed resistors may be nonlinear along three directions by performing multilayer printing and applying a dielectric layer between each resistor print layer. The dielectric layer may withstand the 5 kV defibrillation pulse at the patient end of the resistor, however, along the resistor, the voltage drops gradually, and thus, the dielectric layer at the electronics end layer of the resistor stack may be thinner than that at the patient (5 kV) end.

In some embodiments, maintaining compact resistor size may be beneficial. For example, large resistors may pick up more electromagnetic interference relative to smaller resistors. In addition, for larger resistors, the stray capacitance from the respiration excitation signal line may cause unwanted cross-talk (e.g., signal interference) to the respiration receive line. For the same reasons, it may be beneficial to place the printed resistor at the amplifier end of the wiring, instead of at the electrode end. In some instances, the mentioned cross-talk (e.g., signal interference) between respiration excitation and sense lines may be reduced by using a discrete resistor in the respiration excitation line to reduce the coupling capacitance.

Despite the aforementioned embodiments targeted at improving the printed resistors, resistors may still function sub optimally. The printed resistor may develop a crack due to differential thermal expansion of the resistor material and the substrate, due to local heating and softening of the printed resistor, or due to melting of the substrate. In some instances, the high test voltage may form a localized arc over the crack. In some embodiments, the resistance still limits the current through the wiring because most of the printed resistor is intact.

Furthermore, the resistor may warm evenly due to the defibrillation pulse energy. For example, when the temperature reaches a certain threshold, the binding material or some solvents remaining in the resistor structure may evaporate. The evaporated material, which may include graphite particles, may form a thermally conductive path between the resistor terminals, which may result in an arc between resistor terminals with no current limiting elements present, such that the arc may consume all the defibrillation energy.

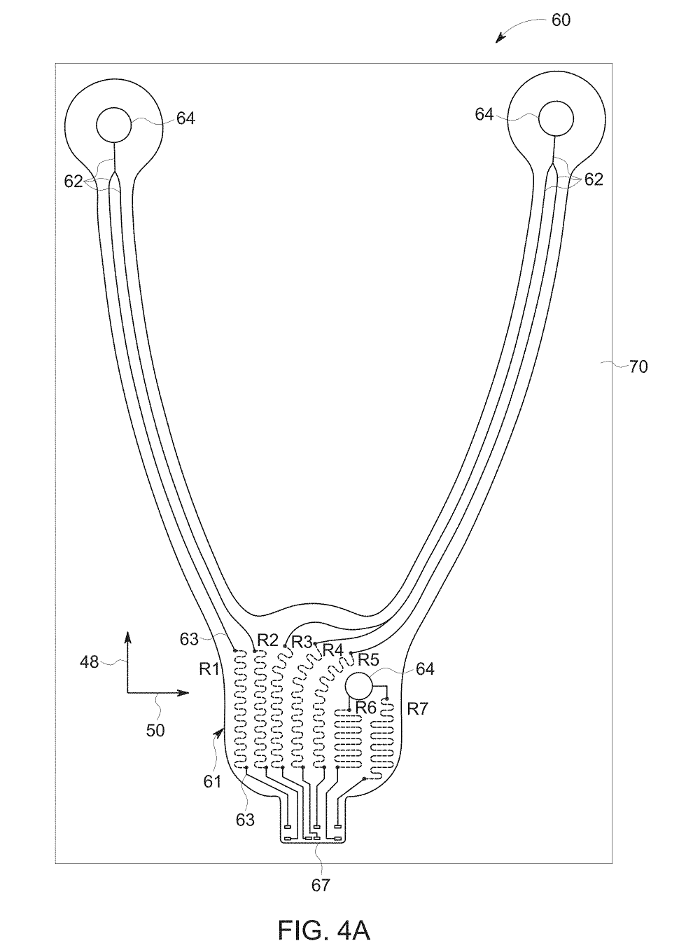

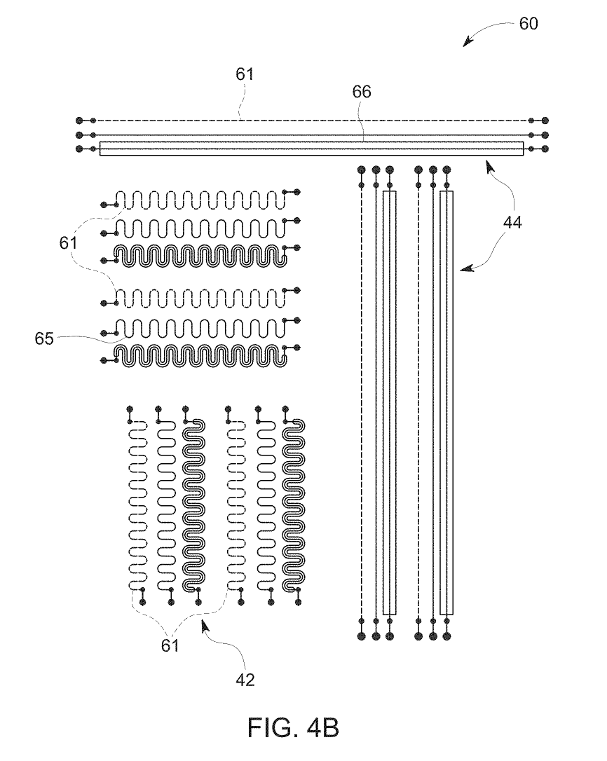

FIG. 4A is a schematic diagram of an embodiment of a printed ECG biopotential sensor 60 and FIG. 4B shows arrangements of printed resistors 61 of FIG. 2, in accordance with aspects of the present disclosure. In some embodiments, the printed ECG biopotential sensor 60 is roll-to-roll (R2R) printed using rotary screen printing to print onto a polymer substrate 70. Thermally conductive inks may be used to print the signal trace 62 and the electrodes 22 (e.g., shown as electrodes 64). In some embodiments, the printed resistors 61, e.g., with a target value of 100 kΩ, are printed using three different inks. The printed electrodes 64 are coupled via the printed single trace 62 to the printed resistors 61. The printed resistors may be coupled to the printed signal trace 62 at one or more electrical contact pads 63. In certain embodiments, the printing of the printed electrodes 64, the printed signal trace 62, and the printed resistors 61 occur during separate printing steps. Accordingly, the printed resistors 61 may be registered to the electrical contact pads. The printed resistors 61 and the signal trace 62 may also electrical couple the sensor 60 to the patient monitor 12 via the monitor end 67. In some embodiments, the printed components may include binding material that causes the printed components to remain intact (e.g., by not shrinking or expanding) on the polymer substrate 70 and to one another in response to a defibrillation pulse.

Furthermore, as shown in FIG. 4B, in some embodiments, the printed resistors 61 may include any suitable number of coating layers (e.g., one, two, three, etc. coating layers) that may dissipate heat from the resistors. The printed resistors 61 may each include any suitable number of resistor layers. For example, a second resistor layer 65 may be printed onto one or more of the resistors 61. (e.g., a first resistor layer, a second resistor layer, etc.). In some embodiments, the resistor layers may include coating layers 66 in between the resistor layers. In certain embodiments, the coating layers 66 may form a heat dissipating, insulating layer over the printed resistor(s) 61. Further, in one embodiment, the coating layers 66 may be registered to the location of the resistors 61 such that the coating layers 66 cover or are positioned in the area corresponding to the resistors 61 and do not cover the entire substrate.

In the illustrated embodiment, the printed ECG biopotential sensor 60 includes multiple straight resistors 44 and nonlinear resistors 42. Further, the printed ECG biopotential sensor 60 includes thermally conductive wiring 62 that connects the electrodes 64 to certain resistors 61. In the illustrated embodiment, the Ag wiring 62 near two of the electrodes splits into two Ag wirings 62, such that the electrodes are each coupled to two resistors 61. In the illustrated embodiment, seven resistors are coupled to the three electrodes 64 via the Ag wiring 62. However, it should be noted that in some embodiments, the Ag wiring 62 may split into any suitable number of Ag wirings 62, and the electrodes may couple to any number of resistors 61. As described in detail below, in the Experimental Results section, resistor test patterns were included in the printed ECG to analyze effects of the resistor layer configuration on the resistance.

### EXAMPLES

### R2R Printing of ECG

The following section introduces experiments and experimental results associated with certain embodiments of the printed ECG biopotential sensor 60. During the experiment, the layers of the printed ECG biopotential sensor 60 were printable and of a suitable layer quality with sharp edges. The layers withstood folding and stretching of the substrate without causing any visually detectable defects (cracks, loss of thermal conductivity, detachment, etc.). The Ag wirings 62 and Ag/AgCl electrodes 64 were thermally conductive and had suitable adhesion to the TPU substrate 70. The printed resistors had rather similar performance, namely a sample-to-sample variation 0.8-1.2% and a resistor-to-resistor variation 5-8%, but had poorer adhesion to the TPU substrate 70 than the silver-based inks. Henkel Resistor inks (which have a resistance of about 200 kΩ) and XCMC-50 resistor inks (which have a resistance of about 150 kΩ) included higher resistance values as the target value of 100 kΩ. However, XRMC-78305 (which have a resistance of about 30 kΩ) ink gave lower resistance values. In one embodiment, the resistance value may be decreased and assessed without compromising the printability by shortening the resistor length (e.g., the layout design), increasing the layer thickness (e.g., by having a coarser screen with lower mesh count), or changing the mixing ratio of the Henkel Resistor ink components. It should be noted that the printability of horizontal lines was of a lesser quality than that of vertical lines.

In one example, the resistors printed using thermally conductive ink, such as Henkel EDAG 6017SS (Henkel AG & Co. KGaA) in combination with Henkel EDAG PM-404 (Henkel AG & Co. KGaA) may form a local crack as described above, for example, due to the use of cellulose nitrate as a carrier substrate. Further, resistors printed using PPG XRMC-50, (PPG Industrial Coatings), include a thermoplastic-based polymer thick film (PTF) carbon conductor ink. In some instances, as polymer carrier heats up, the polymer may get softer, even viscous, instead of forming cracks. This is reflected in the gradual increase in the resistance value when these printed resistors are exposed to consecutive high energy pulses. Furthermore, the resistors printed using PTF carbon conductor ink included resistance values and binding properties favorable for use when exposed to a defibrillation pulse. The resistors printed using PTF carbon conductor ink withstood the high temperatures associated with defibrillation pulses. For example, these printed resistors dissipated heat without delaminating. That is, the printed resistors did not shrink or expand to detach from the substrate they were printed on.

The printing of resistor test patterns was successful. However, as the thickness of the printed stack increased, the runnability and substrate creasing issues became more common. The printed lines became wider as two resistor layers were overprinted due to errors in the manual registration process. Nevertheless, the resistance decreased simultaneously 60-73% down to 64-74 kΩ. Similar resistance decrease was observed when the resistor layers were separated by coating layers. In certain embodiment, registration may be improved by eliminating the substrate shrinking by pre-heating, introducing camera-based registration system, or by increasing the number of registration marks nearer to the printed patterns. During printing, as the ink transfer increased, the edge raggedness and ink spreading increased slightly. The resistor orientation or type (e.g., straight vs. nonlinear) had no effect on the layer thickness or the line width, but horizontal lines had higher resistance than vertical ones. This line dependence decreased when using two resistor layers. The adhesion of the resistor inks to the coating layers was better than to itself or to the TPU substrate. With Henkel Resistor ink the adhesion was the poorest to the TPU substrate whereas with XCMC-50 ink the adhesion was the poorest to itself.

R2R printing experiments were done with Valtion Teknillinen Tutkimuskekus' (VTT's) ROKO printing line using rotary screen printing. The substrate was a 100 μm thick Platilon U073 TPU substrate (Covestro) with a polyethylene terephthalate (PET) carrier foil. The PET carrier foil enabled R2R printing without excessive stretching of the substrate. Five different layers were printed and the printing layout of the layers is shown in FIGS. 4A and 4B. First, the silver (Ag) wirings were printed followed by the Ag/AgCl electrode 64 and resistor layers. Some samples included two coating layers (e.g., insulators) and a second resistor layer to manufacture resistor test structures in the middle of the layout. The test points are presented in Table 1. As used herein and as shown in Table 1, Henkel Resistor refers to a 80:20 Henkel LOCTITE EDAG 6017SS:Henkel LOCTITE EDAG PM-404+8 wt-% of 2-butoxyethyl actate.

TABLE 1
Test points of the ECG demo printings.

| | | | | | 2nd |
|---|---|---|---|---|---|
| Test | Ag | Ag/AgCl | | | Resistor |
| point | wirings | electrode | Resistor | Insulator | layer |
| TP1 | Asahi | ECM | Henkel Resistor* | | |
| | SW1400 | CI-4040 | | | |
| TP2 | ECM | ECM | Henkel Resistor* | | |
| | Cl-1036 | CI-4040 | | | |
| TP3 | Asahi | ECM | PPG XCMC-50 | | |
| | SW1400 | CI-4040 | | | |
| TP4 | ECM | ECM | PPG XCMC-50 | | |
| | Cl-1036 | CI-4040 | | | |
| TP5 | Asahi | ECM | PPG | | |
| | SW1400 | CI-4040 | XRMC-78305 | | |
| TP6 | Asahi | ECM | Henkel Resistor* | 2x Henkel | Henkel |
| | SW1400 | CI-4040 | | EDAG | Resistor* |
| | | | | PF-455B | |
| TP7 | Asahi | ECM | Henkel Resistor* | 2x Henkel | PPG |
| | SW1400 | CI-4040 | | EDAG | XCMC-50 |
| | | | | PF-455B | |

In the illustrated embodiment of FIG. 4, the Ag wirings 62 were printed using Asahi SW1400 and ECM CI-1036 inks. In particular, the inks were rotary screen printed onto the plain TPU substrate 70 at the speed of 2 meters per min (m/min). The mesh count of the screen, in some instances referring to the mesh hole size, (Stork 305V) was 305 threads per inch. The inks were dried at 140° C. in four ovens of ROKO to improve the thermally conductivity. The substrate was not plasma-treated to avoid any substrate changes, such as yellowing. ECM CI-4040 ink was then printed onto the printed wirings to form the Ag/AgCl electrodes 64. The registration was done manually. The printing speed was 2 m/min, the temperature of the ovens was 140° C., and the mesh count of the screen (Stork 305S) was 305 threads per inch.

Three different resistor inks were rotary screen printed to form the printed resistors having the target resistance of 100 kΩ. The resistor layer was manually registered to the Ag wirings 62. Henkel resistor ink was composed of two different resistor pastes in a drying retarding solvent to adjust the layer resistance. Other resistor inks were PPG XCMC-50 and PPG XRMC-78305 carbon inks. All the inks were printed at the speed of 2 m/min and dried at 140° C. in the four ovens of ROKO. The mesh count of the screen (Stork 305S) was 305 threads per inch.

Resistor test structures included resistors having either one or two layers of resistor inks. Accordingly, as provided herein, the resistors may be formed from one or more layers of resistor inks or substances. In some of the test structure, there were two coating layers between the two overprinted resistor layers. The coating layers were printed with Henkel LOCTITE EDAG PF-455B UV-curable insulator ink. The printing speed was 3 m/min and the power level of the UV-curing unit was 50%. The mesh count of the screen (Stork 215V) was 215 threads per inch. The second resistor layer was either printed with Henkel resistor ink or with PPG XCMC-50. The mesh count of the screen (Stork 215V) was 215 threads per inch to be able to produce continuous and/or thermally conductive lines when printing over the edge of the thick coating layers. The printing was done at the speed of 2 m/min and dried at 140° C. The registration of the coating and resistor layers was done manually.

The thickness of the printed layers was measured with Veeco Dektak 150 surface profilometer. The spreading of the Ag wirings 62 and resistors 61 were determined by means of OGP Smartscope 250 microscope. The resistance of the printed layers was measured with a digital multimeter and sheet resistance and volume resistivity values were manually calculated by means of measured data. Ink adhesion to the substrate was analyzed by measuring resistance before and after tape peeling.

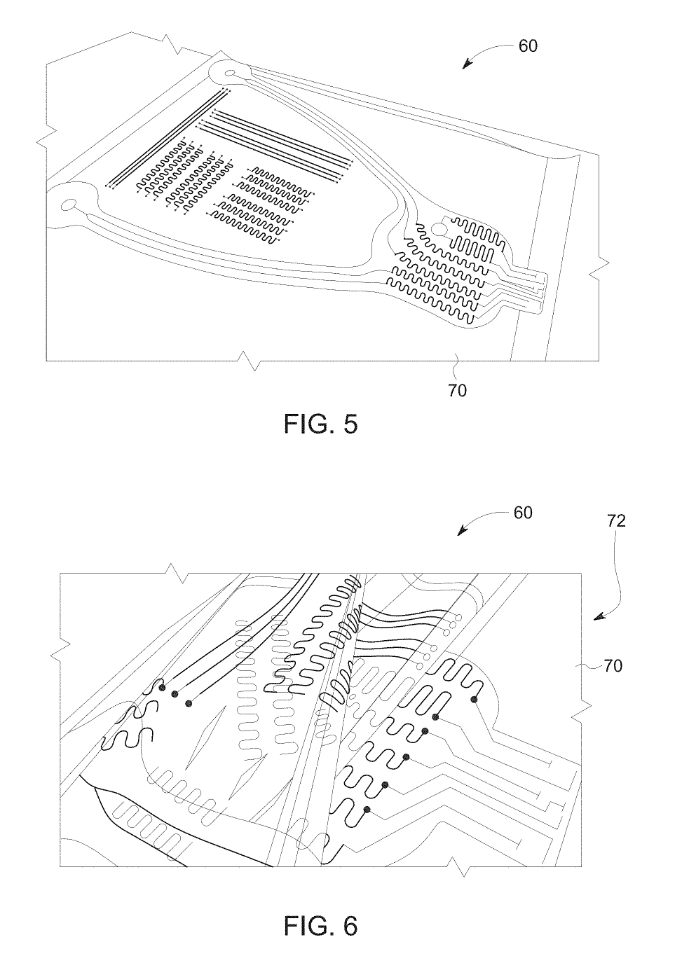

FIGS. 5-8 are embodiments of the printed ECG biopotential sensor 60 of FIG. 4 printed onto the TPU substrate 70, in accordance with aspects of the present disclosure. In the illustrated embodiment of FIG. 5, the printed ECG biopotential sensor 60 includes three different layers printed onto the TPU substrate 70 without any significant issues, such that the illustrated components of the printed ECG biopotential sensor 60 also be registered to each other. Furthermore, FIG. 6 is a schematic diagram 72 another embodiment of the printed ECG biopotential sensor 60 of FIG. 4 printed onto a foldable TPU substrate 70, in accordance with aspects of the present disclosure. As discussed in detail below, the printed ECG biopotential sensor 60 includes various layers that were able to withstand substrate folding and stretching without any visual cracking or detachment from the substrate.

### Ag Wiring

FIG. 7 is a schematic diagram 74 of the Ag wiring 62 printed onto the foldable TPU substrate 70 of FIG. 6, in accordance with aspects of the present disclosure. The printability and visual print quality of the Ag wirings 62 is suitable for ECG applications. In the illustrated embodiment, the inks are properly transferred onto the TPU substrate 70 and form uniform layers with sharp edges. The print quality of Ag wire 62 printed with Asahi SW1400 and ECM CI-1036 inks was compared, and the layer properties are shown in Table 2.

TABLE 2
Layer properties of the printed silver wirings.

| | | Line | Sheet | Volume | Adhesion- |
|---|---|---|---|---|---|
| | Thickness | width | resistance | resistivity | change in |
| | (μm) | (mm) | (mΩ/□) | (Ω · cm) | resistance (%) |
| SW1400 | 5.8 | 1.17 | 139 | 8.4E−05 | 2.0% |
| CI-1036 | 4.2 | 1.18 | 273 | 1.1E−04 | 4.4% |

As shown in Table 2, the Asahi SW1400 ink produced thicker layers with better thermal conductivity than ECM CI-1036 ink probably due to the higher silver loading of SW1400. In addition, the adhesion appeared to be slightly better with SW1400. No significant differences were seen in the ink spreading between the silver inks. The thermally conductivity on the TPU substrate 70 are lower than when printed onto the PET substrate. The difference is explained by the thin layer and high substrate roughness.

### Ag/AgCl Electrodes

The printing process of the Ag/AgCl ink was smooth. The dimensions of the substrate slightly changed during the printing of the Ag wirings 62 as a result of registration. In certain embodiments, pre-heating of the substrate prior to any printing may improve the registration accuracy by causing any potential the dimensional changes of the substrate (shrinking, elongation) before any layer is printed. The Ag/AgCl electrode 64 (FIG. 4) layer had good coverage and sharp edges. Furthermore, the Ag/AgCl electrodes 64 were rotary printed using ECM CI-4040 ink. The layer properties of the Ag/AgCl electrodes 64 are presented in

Table 3. The ECM CI-4040 layer is thermally conductive and its properties are rather close to those of the Ag wirings 62. The layer also had good adhesion to the substrate.

TABLE 3
Layer properties of Ag/AgCl electrodes

| | | Sheet | Volume | Adhesion - |
|---|---|---|---|---|
| | Thickness | resistance | resistivity | change in |
| | (μm) | (mΩ/□) | (Ω · cm) | resistance (%) |
| CI-4040 | 6.4 | 260 | 1.7E−4 | 2.6% |

### Resistors

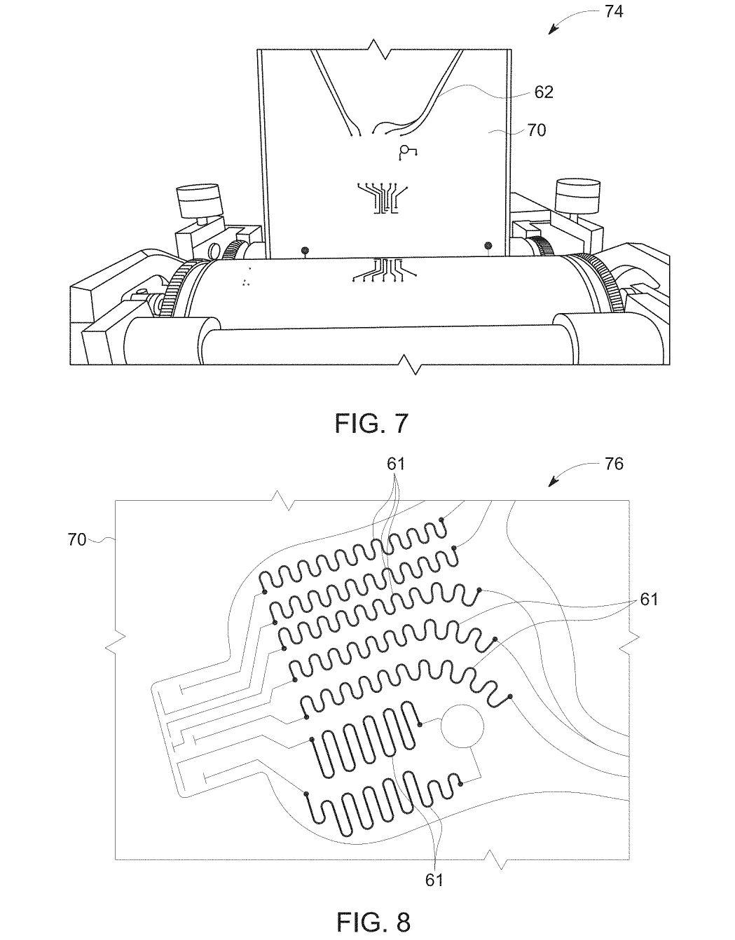

FIG. 8 is a schematic diagram 76 of another embodiment of the variety of printed resistors of FIG. 2 included in the ECG biopotential sensor 60 of FIG. 4 printed onto the foldable TPU substrate 70 of FIG. 6, in accordance with aspects of the present disclosure. During the experiment, three resistor inks, namely, Henkel Resistor ink, PPG XCMC-50 ink, and PPG XRMC-78305 ink were used and compared. The three resistor inks are properly printable and they result in suitable print quality. The resistor layers had excellent coverage (e.g., without pinholes) and sharp, well-defined edges. PPG XRMC-78305 appeared to reproduce slightly more ragged edges resulting from its slower evening out inside the rotating screen. All the layers seem to remain on the substrate 70 during folding and heavy manual stretching of the substrate 70, such that the resistor layer is not delaminated from the substrate 70 or cracked in an amount to lose its thermal conductivity completely. The properties of the printed resistor layers are shown in Table 4.

TABLE 4
Layer properties of the ECG resistors.

| | | Line | | |
|---|---|---|---|---|
| | Thickness | width | Resistance (kΩ) - | Adhesion - change |
| | (μm) | (mm) | Target 100 kΩ | in resistance (%) |
| Henkel | 4.3 | 0.98 | 201.7 ± 3.9% (TP1) | 32% |
| resistor | | | 182.1 ± 3.8% (TP2) | |
| XCMC-50 | 5.9 | 1.01 | 150.2 ± 6.4% (TP3) | 15% |
| | | | 150.3 ± 7.1% (TP4) | |
| XRMC- | 5.9 | 0.99 | 31.6 ± 4.6% (TP5) | 48% |
| 78305 | | | | |

As shown in Table 4, the Henkel resistor is thinner than PPG resistors resulting from the added co-solvent (e.g., reduces solids content). The resistor values of the Henkel Resistor and the PPG XCMC-50 are higher than the target value of 100 kΩ. The XRMC-78305 ink produces lower resistance values. The resistance values of the Henkel Resistor and the PPG XCMC-50 inks may be decreased by shortening the resistor length (layout design), increasing the layer thickness (e.g., coarser screen with lower mesh count), or by changing the mixing ratio of the Henkel Resistor ink components. The increase in the resistance value of PPG XRMC-78305 ink may be achieved by adding co-solvents into the ink. The standard deviation of the resistance values is less than 5% in the case of the Henkel Resistor ink and the XRMC-78305 ink, and less than 8% with the XCMC-50 ink. Typically, the variation of a single resistor is 0.8-1.2% but the resistance variations between the resistors are increasing the deviations. The adhesion of XCMC-50 appeared to be the best, but no significant visual differences in the amount of detached ink amount was visible. The XRMC-78305 ink appeared to be detached the most during the tape peeling test.

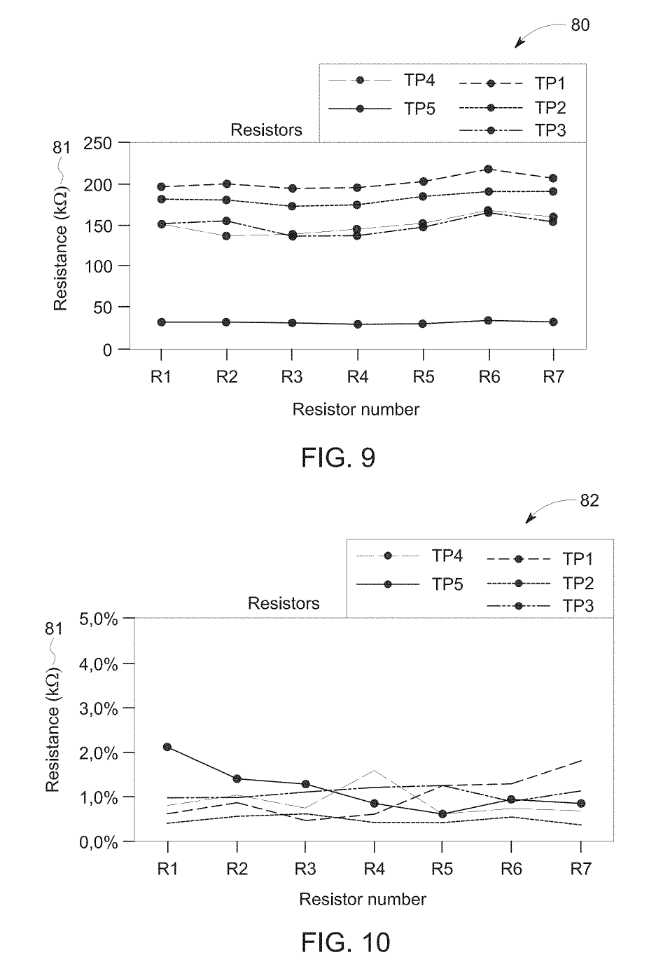

FIG. 9 is a line graph 80 of the resistance 81 (in kΩ) for various resistors corresponding to various test points, in accordance with aspects of the present disclosure, and FIG. 10 is another line graph 82 of the deviation for the various resistors corresponding to the various test points, in accordance with aspects of the present disclosure. In particular, FIG. 9 depicts the resistance 81 against the resistor numbers for the test points of Table 1, and FIG. 10 depicts the resistor deviation against the resistor numbers for the test points of Table 1. In the illustrated embodiment, the effect of the resistor ink and resistor placement on the resistance 81 and its deviation are shown. The resistors R6 and R7 (e.g., the resistors with the widest nonlinear patterns) seem to have slightly higher resistance values than the other resistors. The Henkel Resistor also gives different resistance values between test points (e.g., TP1 vs. TP2), thus indicating changes in the ink layer during the print run or some differences in printability onto different Ag wirings. The resistor deviations are less than 2%, so the print quality remained uniform from sample-to-sample.

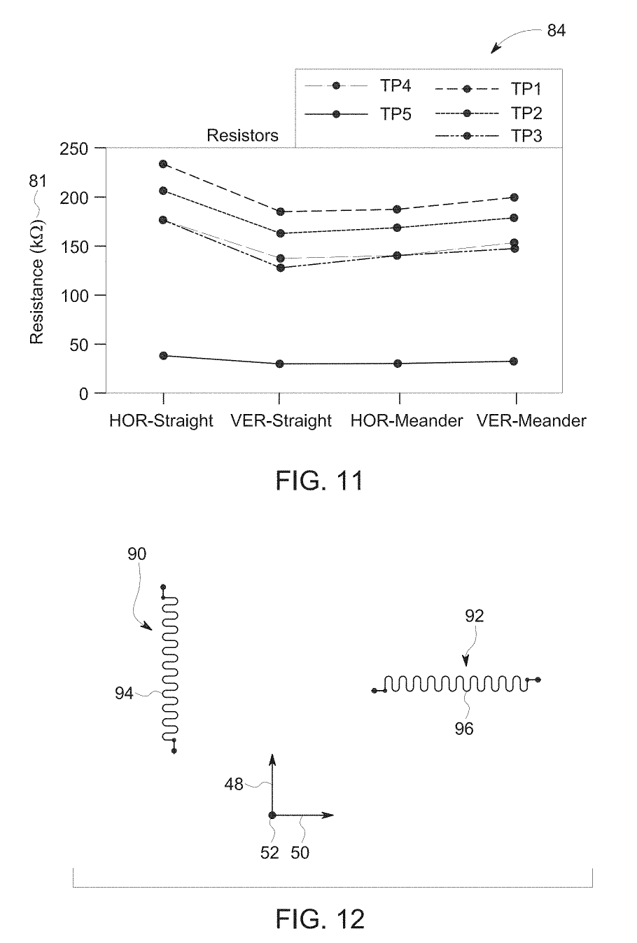

FIG. 11 is another line graph 84 of the resistance 81 for various resistor placements, resistor types, and inks corresponding to various test patterns, in accordance with aspects of the present disclosure. In other words, the effect of the resistor ink and resistor type and orientation are presented in FIG. 11. FIG. 12 is a schematic diagram of an embodiment of a vertical nonlinear resistor 90 and a horizontal nonlinear resistor 92 of the variety of printed resistors of FIG. 2, in accordance with aspects of the present disclosure. In the illustrated embodiment, the vertical nonlinear resistor 90 is aligned along the first direction 48 and includes horizontal lines 94, while the horizontal nonlinear resistor 92 is aligned along the second direction and includes vertical lines 96. The horizontal lines 94 have higher resistance than vertical lines 96 resulting from the poorer printability of the horizontal lines 94. The horizontal nonlinear resistors 92 have lower resistance values than straight resistors, whereas the opposite applies to vertical nonlinear resistors 90. This results from the fact that vertical nonlinear resistors 90 include more horizontal lines 94 having the poorer printability.

### Resistor Test Patterns

The printing of the coating layer and the second resistor layer onto the resistor test patterns was also successful. Due to the high thickness of the coating layer and the fact that only small parts of the substrate are covered with it, creases are easily formed when re-winding the substrate at the end of the machine. This may cause changes in the underlying printed layer properties, such as thermal conductivity. However, the printing processes of the second coating layer and resistor layer are not affected.

The print quality of different resistor test structures was compared. Specifically, a first test structure including, one layer Henkel Resistor ink (1× Henkel Resistor) printed on a plain TPU substrate 70, a second test structure including two layer Henkel Resistor ink (2× Henkel Resistor) that have been overprinted, a third test structure including one Henkel Resistor ink layer (1× Henkel Resistor-2× Insulator layer-1× Henkel Resistor) printed onto two coating layers, a fourth test structure including one layer XCMC-50 ink (1× XCMC-50) printed on a plain TPU substrate 70, a fifth test structure including two layer XCMC-50 ink (2× XCMC-50) that has been overprinted, and a sixth test structure (1× XCMC-50-2× insulator layers-1× XCMC-50) printed onto two coating layers.

When a single resistor layer is printed onto the TPU substrate 70, the printed edges are well-defined and sharp. As two layers are overprinted, the printed lines are wider and the printed edges are more ragged. This results from the higher ink transfer amount of the second resistor layer and the registration errors. When the resistor layer is printed onto coating layers, the printed edges are ragged, but the line width is not visually affected. Therefore, the main explanation for the wider lines of the double resistor layer comes from the errors in the manual registration process (e.g., done visually during printing). The registration accuracy may be improved by pre-heating the substrate, acquiring some camera-based registration system (MAXI line at VTT has such a system, but poorer TPU runnability), or improving the number and quality of the registration marks on the layout. The properties of the printed resistor and coating layers (e.g., insulator) are shown in Table 5.

TABLE 5
Properties of the printed resistor and coating layers
(e.g., insulators).

| | | | Line | |
|---|---|---|---|---|
| | | Thickness | width | Adhesion - change |
| Ink | Layer | (μm) | (mm) | in resistance (%) |
| Henkel resistor | 1^{st} layer | 4.0 | 0.98 | 32% |
| 2xHenkel | 1^{st} and 2^{nd} layer | 9.4 | 1.14 | 10% |
| resistor | | | | |
| Henkel resistor | 2^{nd} layer on | 5.0 | 1.00 | 5% |
| | insulator | | | |
| XCMC-50 | 1^{st} layer | 5.8 | 1.00 | 15% |
| 2xXCMC-50 | 1^{st} and 2^{nd} layer | 11.2 | 1.15 | 24% |
| XCMC-50 | 2^{nd} layer on | 10.0 | 1.03 | 5% |
| | insulator | | | |
| 2xPF-455B | Insulator | 32.7 | — | — |

The coating layers are thick since the ink has 100% solid content and is UV-curable. As a result, no material is removed from the printed layer during curing. When two layers of resistor inks are overprinted the layer thickness increases, but this also increases the line width due to registration errors and increased amount of ink on the substrate. The second resistor layer on the top of the coating layer is thicker than the bottom resistor layer resulting from the coarser screen that increased the ink transfer. This also increases the line width slightly. The resistor orientation or type has no effect on the layer thickness or the line width. The adhesion of the resistor inks to the coating layers is better than to itself or to the TPU substrate 70. With Henkel Resistor ink, the adhesion is the poorest to the TPU substrate 70, whereas with XCMC-50 ink the adhesion is the poorest to itself.

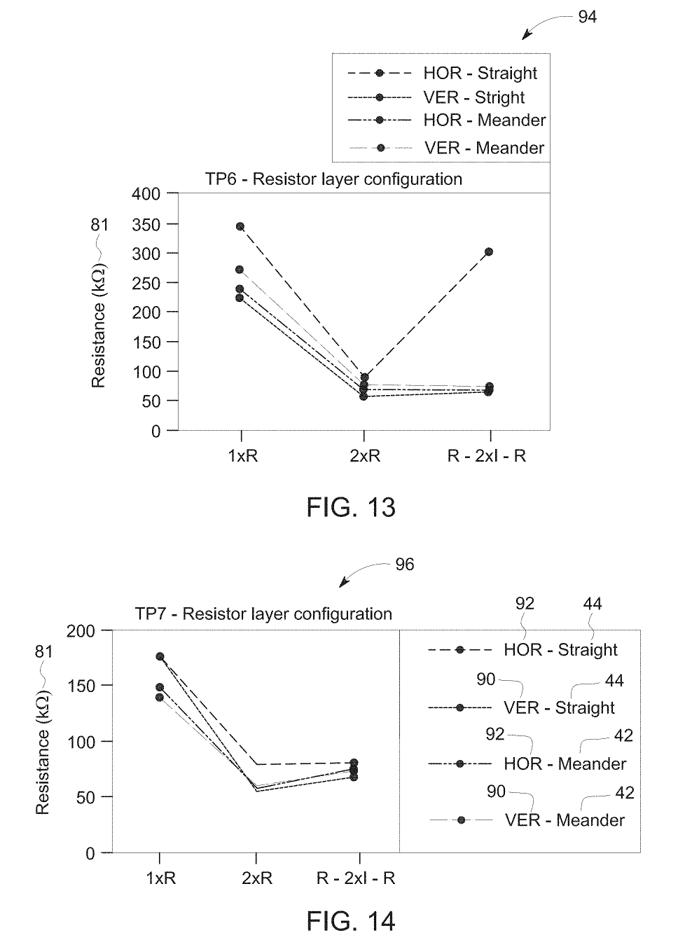

FIG. 13 is a line graph 94 of the resistance 81 against the resistor orientation and resistor layer configuration for the Henkel Resistor ink, in accordance with aspects of the present disclosure. FIG. 14 is a line graph 96 of the resistance 81 against the resistor orientation and resistor layer configuration for the PPG XCMC-50 ink, in accordance with aspects of the present disclosure. In the illustrated embodiment, the resistor orientation is designated with HOR (referring to horizontal resistors 92) or VER (referring to vertical resistors 90). Further, in the illustrated embodiment, the type of the resistor is designated with straight (referring to straight resistors 44) or nonlinear (referring to nonlinear resistors 42). In addition, the resistor layer configuration is designated as 1×R (referring to one bottom resistor layer), 2×R (referring to a bottom resistor layer and a top resistor layer), or R-2×I-R (referring to a bottom resistor and a top resistor having two coating layers in between).

The effect of the resistor orientation and type as well as the resistor layer configuration on the resistance values, are shown in the line graphs 94 and 96. When two layers of resistor ink are overprinted, the resistance decreases 60-73%. The Henkel Resistor values drop from 270 kΩ to 74 kΩ and the XCMC-50 values drop from 160 kΩ to 64 kΩ. When the two resistor layers are printed such that two coating layers separate them (except at the contact points to the Ag wirings 62), the resistance values are very close to the values when two resistor layers are overprinted. The exception is seen with Henkel Resistor ink when using horizontal straight resistors because the used screen was clogged and/or not opened during the patterning process of the screen from that exact area (e.g., not continuous line). The Henkel Resistor ink creates higher resistance values with horizontal straight resistors and vertical nonlinear resistors than with other resistors whereas with XCMC-50 ink straight resistors have higher values than nonlinear resistors. As two layers are overprinted, the effect of the resistor type and orientation decreases. However, horizontal straight resistors create still higher resistance values.

### R2R Print Quality of Respiratory Prototypes and Resistors Onto TPU Substrate

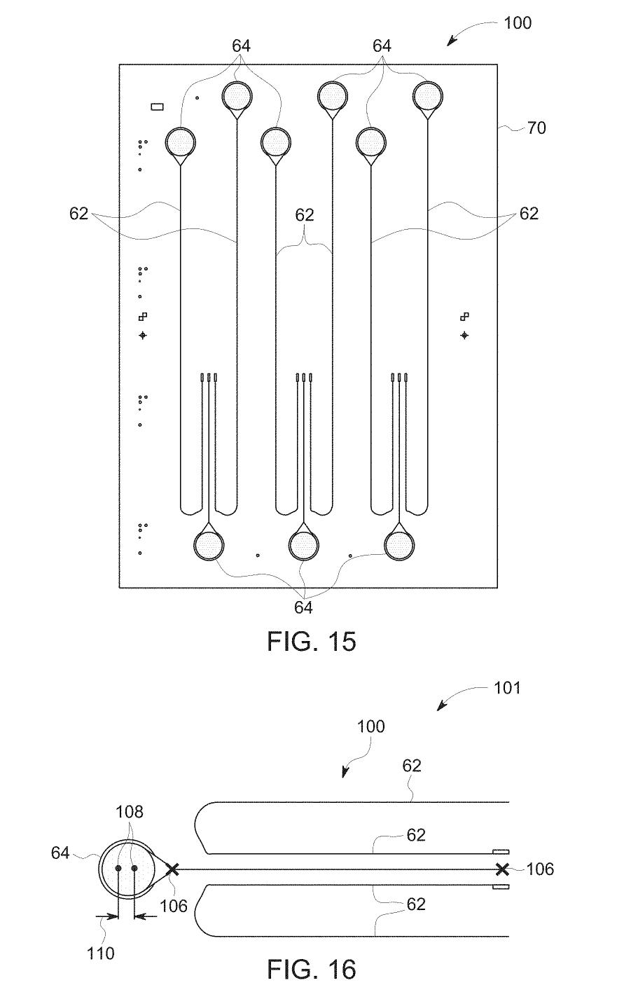

FIG. 15 is a schematic diagram of an embodiment of a printed respiratory prototype 100, in accordance with aspects of the present disclosure. As mentioned above, any suitable biopotential sensor, such as a blood pressure sensor, an ECG sensor, a pulse oximeter sensor, an electromyography sensor, an inertial sensor, a respiratory sensor, or the like, may be printed using the embodiments disclosed herein. The respiratory prototype 100 may be printed using rotary screen printing onto a stretchable TPU substrate 70. First, Ag wirings 62 may be printed using two different silver pastes after which the Ag/AgCl electrodes 64 may be printed and registered on top. In some embodiments, the layers may easily register to each other, such that no additional substrate deformations take place after the pre-heating step of the TPU substrate 70. The Ag wiring and the Ag/AgCl electrode layers may be printed using the embodiments disclosed herein, such that a suitable layer quality is achieved. Furthermore, the printed respiratory prototype 100 may include any suitable printed resistors of any suitable shapes and sizes. For example, the printed respiratory prototype 100 may include straight resistors 44, nonlinear resistors 42, resistors printed with Henkel ink, resistors printed on the TPU substrate 70, resistors oriented in specific locations on the substrate, and the like.

As discussed, when experiments to print the respiratory prototype 100 were executed, the Ag wirings 62 suffered slightly from the side-dependence of the resistance value that might result from the printing parameters or screen quality. However, the visual print quality was not affected. Furthermore, the Ag/AgCl electrode layer had uneven layer quality at some locations due to the poor quality of the screens by the sub-contractor. All the printed layers withstood bending, handling, and slight stretching of the substrate well, thus making them potential materials for various applications and to be used with stretchable substrates.

All the resistor inks were properly printable with good coverage. The Henkel ink gave the resistance values closest to the target values. Nonlinear resistors values were on average 3% larger (e.g., range: −1%-10%) than the target values. Straight resistor values were 2% larger but the range was larger (e.g., −10%-13%), thus making nonlinear resistor shapes slightly more preferable. The resistor shape (e.g., nonlinear vs. straight) did not affect the sample-to-sample resistance deviations much with the Henkel ink. The average resistance deviation was 2.5% (e.g., maximum 5.4%) and 2.3% (maximum 6.9%) for nonlinear and straight resistors, respectively. Other tested resistor inks gave much higher sample-to-sample deviations with straight resistors than with nonlinear resistors. As a conclusion, based on the experimental data, the nonlinear resistors 42 (FIG. 4) seem to give more even resistance values that are closer to the target values compared to the straight resistors 44 (FIG. 4). However, it should be noted that the resistor evenness may depend on the orientation of the resistors on the screen. For example, vertically aligned straight resistors, as used in this experiment, might give more even print quality than horizontally aligned ones, although in previous printing tests the orientation did not significantly affect the deviation of the resistance value. It should be noted that only Henkel ink was printed from a “fresh” jar (hereinafter referring to a new jar, including new material, opened for the first time for testing purposes). Further, since the delivery of the PPG inks was delayed, “old” material (hereinafter referring to material from a jar previously opened) had to be used. This may have had some effect on the results and they should be confirmed in the next sheet-to-sheet printing tests in which “fresh” material will be used. It is also possible that higher deviations in resistance values of straight waveguides is due to stretching of the TPU substrate. Further, nonlinear resistors 42 withstand stretching inherently better than straight resistors 44 and it is possible that this phenomenon is seen with PPG inks.

More even resistance values were obtained when the resistor lines were wider and longer. A line width of 1.5 mm seemed to result in resistance values that are nearest to the target values. Straight resistors were more unevenly reproduced (e.g., larger sample-to-sample deviation) than other shapes. The increase in the resistor length (e.g., constant line width) increased the resistance value, and slightly smaller deviation was seen with straight resistors longer than 4 mm. It might be beneficial to avoid short resistors having narrow line widths in the future to keep the sample-to-sample deviations smaller. The width of the end part of the resistor lines had no effect on the resistor evenness but target values were the closest when the end width was 2 or 3 mm with 1 mm resistor line widths.

The respiratory prototypes were manufactured with VTT's ROKO R2R printing line using rotary screen printing onto a stretchable TPU substrate 70. The TPU substrate 70 was Covestro Platilon U073 and was 100 μm thick. The printing step was done in the first printing unit after which the drying took place inline in the four ovens of ROKO. The TPU substrate 70 had a PET carrier foil to avoid stretching or shrinkage of the substrate during the R2R printing and drying processes under web tension and to allow steady substrate travel in the printing unit. To enable multilayer printing having good layer-to-layer register, the TPU substrate 70 was pre-heated prior to the actual printing steps. As a result, any small substrate deformations took place before anything was printed onto the substrate. The pre-heating speed was 5 m/min and the temperature of the ovens were 140° C.

The printing layouts of the respiratory prototypes 100 is shown in FIG. 15. The Ag wirings 62 were printed with Asahi SW1400 or ECM CI-1036 silver pastes onto the TPU substrate 70. The printing speed was 2 m/min and the temperature of the ovens was 140° C. The screen type was Stork 305V (e.g., 305L line density, 15 μm wet ink layer thickness, 22% open area). The Ag/AgCl electrodes 64 were then printed onto the Ag wirings 62 using EMC CI-4040 paste. The printing speed was 2 m/min, the drying temperature 140° C., and the screen type Stork 305S (305L, 11 μm, 21%). The electrode layer was manually registered to the underlying Ag wirings 62.

FIG. 16 is a schematic diagram of an embodiment of a portion 101 of the printed respiratory prototype 100 of FIG. 15 and testing measurement locations 106, in accordance with aspects of the present disclosure. The printed samples were analyzed. Specifically, five parallel samples were measured from several different locations. The thickness of the printed layers was measured with Veeco Dektak 150 surface profilometer. The print quality of the Ag wirings 62 and the electrodes 64, and the spreading of the Ag wirings 62 was determined by means of OGP Smartscope 250 microscope. The magnification was ×37.7. The resistance of the printed layers was measured with a digital multimeter. The sheet resistance and volume resistivity values of the layers were then calculated by means of the measured line width and layer thickness data. During the measurement process, the various multimeter measurements were taken at the Ag wiring measurement locations 106 and at the electrode measurement locations 108. The measuring distance 100 between the two electrode measurement locations 108 was about one centimeter, and the resistance between the two electrode measurement locations 108 was noted. Further, the resistance between the two Ag wiring measurement locations 106 was also noted.

### Ag Wiring

Ag wirings 62 were printed with rotary screen printing onto the TPU substrate 70. The printing process was smooth with both of the inks. Both of the Ag paste layers had good coverage. However, the CI-1036 layers have some edge raggedness. Table 6 presents the layer properties of the printed Ag wirings and Table 7 presents the effects of the placement of the Ag wiring 62 on the TPU substrate 70 on the layer properties. In Table 7 and as used herein “MS” refers to the machine-side, “US” refers to the user-side, and “CENTER” refers to the center of the substrate.

TABLE 6
Layer properties of the printed Ag wirings.

| | | | Square | Volume |
|---|---|---|---|---|
| | Thickness | Line width | resistance | resistivity |
| Ink | (μm) | (μm) | (mΩ/unit area | (Ω · cm) |
| SW1400 | 6.58 ± 1.02 | 780 | 107 | 7.0E−05 |
| CI-1036 | 5.35 ± 0.93 | 774 | 77 | 4.1E−05 |

TABLE 7
Effect of the placement of the Ag wiring (on the substrate) on the layer properties.

| Ink | Thickness (μm) | | | Resistance (Ω) | | | Line width (μm) | | |
|---|---|---|---|---|---|---|---|---|---|
| SW1400 | 6.99 | 6.46 | 6.28 | 14.2 ± 1.6% | 14.9 ± 2.4% | 17.6 ± 4.6% | 780 | 783 | 778 |
| CI-1036 | 5.58 | 5.16 | 5.30 | 10.6 ± 0.8% | 11.2 ± 1.5% | 12.4 ± 2.6% | 769 | 770 | 783 |

As shown in Tables 6 and 7, the SW1400 ink layer is thicker, but the thermal conductivity is slightly lower than that of the CI-1036 layer. The edge raggedness of CI-1036 wirings seem to result from the slightly lower ink spreading. There are some side dependence in layer properties with both of the inks, since the resistance values of the wirings and their deviations increase from the MS to the US of ROKO, as shown in Table 7. With SW1400 ink, the layer thickness also decreases from MS to US, thus indicating ink transfer unevenness. With CI-1036 ink, the layer thickness is the smallest in the middle but the ink layer spreads the most in the US of the substrate. Both of these factors indicate some unevenness in the ink transfer and can lead to increased resistance values. The factors affecting this side dependence are uneven squeegee pressure or unevenly worn squeegee, variating squeegee angle, uneven thickness of the emulsion from the screen manufacturing, uneven web tension during the ink transfer process in the nip, uneven ink flow, and/or the amount inside the rotating screen. However, any visual difference between the MS and US wirings was not seen.

### Ag/AgCl Electrodes

The Ag/AgCl electrodes 64 were printed onto the Ag wirings 62 with ROKO using rotary screen printing. The electrode layer was manually registered to the wiring layer. Because of the pre-heating treatment of the substrate prior to any printing, the substrate did not deform during the printing and drying of the Ag wirings 62, thus enabling easy and good registration of the electrode layer. The print quality of the Ag/AgCl layer (CI-4040) was good with sharp edges. However, the screens (e.g., acquired from a sub-contractor) were initially partly clogged which affects the electrode coverage and evenness. These defects result from the poor washing evenness of the screens after the exposure through the page film. The layer properties of CI-4040 layer are presented in Table 8. Based on Table 8, slightly thicker layer but higher resistance value is achieved when printing CI-4040 ink onto SW1400 wirings than onto CI-1036 wirings.

TABLE 8
Layer properties of Ag/AgCl electrodes printed with
CI-4040 paste

| CI-4040 on wirings | Thickness (μm) | Resistance - 1 cm distance (Ω) |
|---|---|---|
| SW1400 | 7.65 ± 1.68 | 0.60 |
| CI-1036 | 6.53 ± 1.42 | 0.47 |

### R2R Printed Resistor

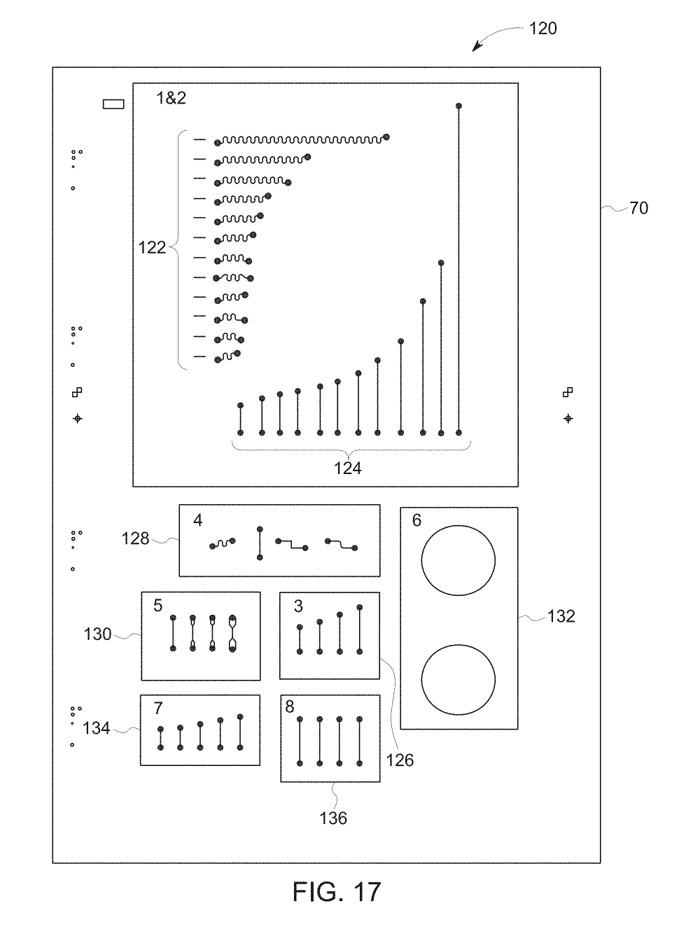

FIG. 17 is a schematic diagram of an embodiment of printed resistors 120 employed in the printed respiratory prototype 100 of FIG. 15, in accordance with aspects of the present disclosure. In particular, during the experiment, the illustrated resistor test patterns were printed onto the TPU substrate 70 (e.g., Covestro Platilon U073) with rotary screen printing in ROKO. In the illustrated embodiment, six test patterns for the printed resistors were printed onto the TPU substrate 70. In the illustrated embodiment, a first test pattern 122 includes nonlinear resistors 42 of various sizes corresponding to certain resistances, namely, 10 kΩ, 15 kΩ, 17 kΩ, 20 kΩ, 23 kΩ, 25 kΩ, 30 kΩ, 40 kΩ, 50 kΩ, 75 kΩ, 100 kΩ, and 200 kΩ. In the illustrated embodiment, a second test pattern 124 included straight resistors 44 of various sizes corresponding to certain resistances, namely, 10 kΩ, 15 kΩ, 17 kΩ, 20 kΩ, 23 kΩ, 25 kΩ, 30 kΩ, 40 kΩ, 50 kΩ, 75 kΩ, 100 kΩ, and 200 kΩ. In the illustrated embodiment, a third test pattern 126 included 10 kΩ having different line widths, namely, 0.5 mm, 1 mm, 1.5 mm, and 2 mm line widths. In the illustrated embodiment, a fourth test pattern 128 included 10 kΩ resistors having different shapes, namely, a nonlinear resistor, a straight resistor, a sharp stepped resistor, and a soft stepped resistor. In the illustrated embodiment, a fifth test pattern 130 included 10 kΩ resistors having different line widths near the contact pads, namely, 1, 2, 3, and 4 mm line widths. In the illustrated embodiment, a sixth test pattern 132 included the test patterns for the thermal conduction measurements. In the illustrated embodiment, a seventh test pattern 134 included resistors having different lengths, namely, 1 mm, 2 mm, 4 mm, 6 mm, and 8 mm. In the illustrated embodiment, an eight test pattern 136 includes the illustrated resistor patterns.

During the experiment, the TPU substrate 70 was pre-heated (e.g., at 5 m/min at 140° C.) before any printings to allow accurate multilayer printing. Three different resistor inks were printed onto the TPU substrate 70 at the speed of 2 m/min and dried at 140° C. The screen type was Stork 305S (e.g., 305L, 11 μm, 21%). The inks were Henkel resistor (e.g., 80:20 wt-% Henkel EDAG 6017SS:Henkel EDAG PM-404+8 wt-% BGA co-solvent), PPG XCMC-50, and PPG XRMC-78305 carbon pastes. Then the contact pads were printed onto the resistors and registered manually to the resistors. Asahi SW1400 silver paste was printed at the speed of 2 m/min and dried at 140° C. The screen type was Stork 305V (305L, 15 μm, 22%).

Five (5) parallel samples were analyzed. The thickness of the resistor layers was measured with Veeco Dektak 150 surface profilometer. The spreading and visual quality of the resistors were determined by means of OGP Smartscope 250 microscope. The resistance of the printed layers was measured with a digital multimeter between contact pads. Sheet resistance and volume resistivity values were calculated by means of the measured line width and layer thickness data.

### Layer Quality

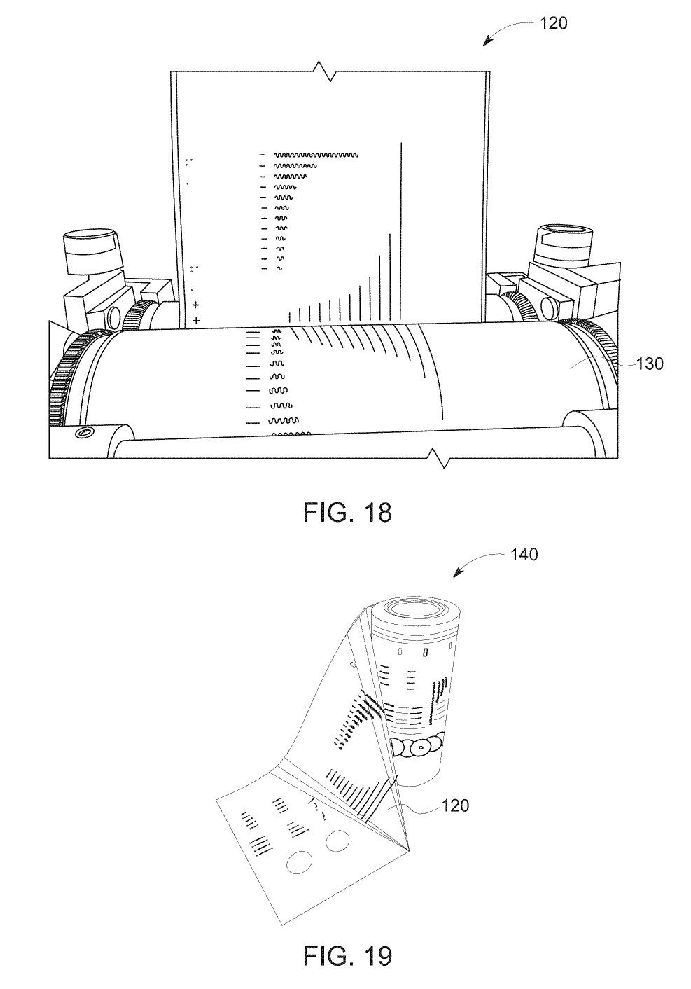

FIG. 18 is a schematic diagram of an embodiment of a printer 130 configured to print the printed resistors 120 of FIG. 17, in accordance with aspects of the present disclosure. In some embodiments, the process of printing the printed resistors may employ carbon pastes and contact pads using silver paste. In some embodiments, the printed resistors and thermally conductive inks are printed using rotary screen printing. In some embodiments, the registration process is done manually and visually due to which some registration errors occur occasionally in the printed rolls. In the future, the registration errors could most likely be eliminated by using camera-based automatic registration systems that can detect smaller registration errors not visible by the human eye. The substrate deformations are mainly eliminated in the pre-heating step of the substrate. As a result, the printing unit parameters can be kept constant from layer-to-layer and no registration difficulties occur. FIG. 19 is a schematic diagram 140 of an embodiment of the printed resistors 120 of FIG. 17, in accordance with aspects of the present disclosure. In particular, the printed resistors 120 may be printed on a foldable and stretchable TPU substrate. In some embodiments, the resistors printed using carbon-based conductor ink included binding properties favorable for use when exposed to a defibrillation pulse. For example, these printed resistors may dissipate heat without delaminating. That is, in such embodiments, the printed resistors do not shrink or expand to detached from the substrate.

### Effect of the Ink and Resistor Size

The resistor layers had well-defined edges and good coverage. The XRMC-78305 ink layer appeared to have some edge raggedness in the curved parts of the patterns. The Henkel ink layer was the thinnest and it spread the least. The XRMC-78305 ink layer was the thickest and had the highest thermal conductivity. The properties of the three resistors is shown in Table 9.

TABLE 9
Resistor layer properties.

| | | Line width | Square | Volume |
|---|---|---|---|---|
| | | of 1 mm line | resistance | resistivity |
| Ink | Thickness (μm) | (μm) | (Ω/□) | (Ω · cm) |
| Henkel | 3.80 ± 0.95 | 956 | 1120 | 2.9E−01 |
| XCMC-50 | 6.26 ± 1.24 | 987 | 890 | 4.6E−01 |
| XRMC-78305 | 7.74 ± 0.79 | 982 | 196 | 1.0E−01 |

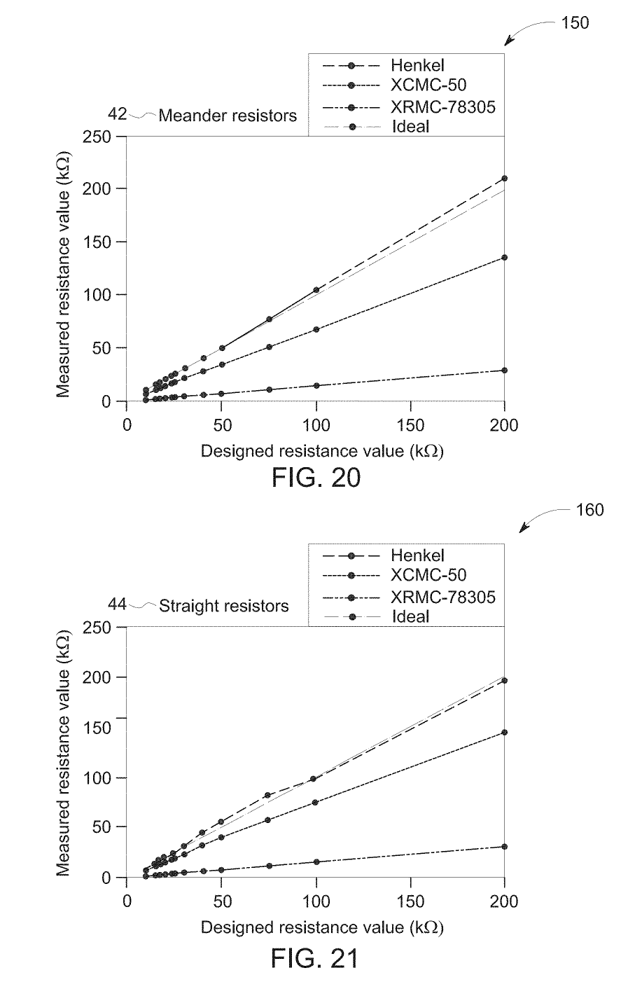

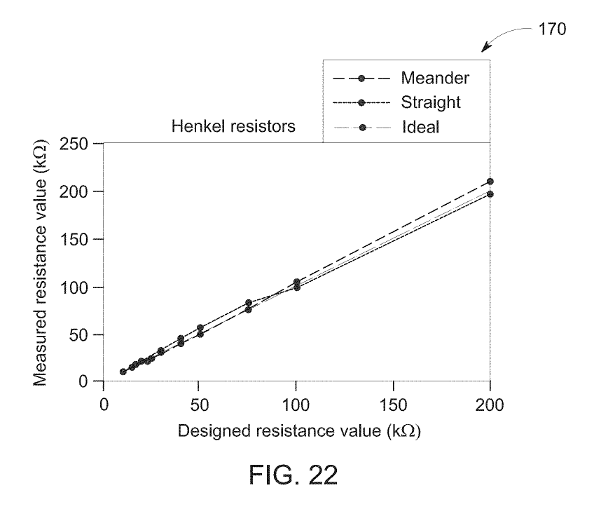

FIG. 20 is a line graph 150 of the measured resistance value of nonlinear resistors 42 having various resistance values corresponding to various resistor inks, in accordance with aspects of the present disclosure. Furthermore, FIG. 21 is a line graph 160 of the measured resistance value of straight resistors 44 having various resistance values corresponding to various resistor inks, in accordance with aspects of the present disclosure. FIG. 22 is a line graph 170 of the measured resistance value of the Henkel resistors of FIG. 13 having various resistance values corresponding to various resistor inks, in accordance with aspects of the present disclosure. Further, the measured resistance values of the different resistor inks and different resistor shapes are presented in Table 10.

TABLE 10
Measured resistance values of printed straight
and nonlinear resistors using different inks.

| De- | | | | | | |
|---|---|---|---|---|---|---|
| signed | Measured resistance - meander | | | Measured resistance - straight | | |
| resis- | (kΩ) | | | (kΩ) | | |
| tance | | XCMC- | XRMC- | | XCMC- | XRMC- |
| (kΩ) | Henkel | 50 | 78305 | Henkel | 50 | 78305 |
| 200 | 209.8 | 135.0 | 28.5 | 196.2 | 145.1 | 33.3 |
| 100 | 104.3 | 67.3 | 14.3 | 98.3 | 74.1 | 16.6 |
| 75 | 75.8 | 51.1 | 10.6 | 82.3 | 57.1 | 12.8 |
| 50 | 49.5 | 33.9 | 6.8 | 56.0 | 39.6 | 8.3 |
| 40 | 40.1 | 27.8 | 5.5 | 45.3 | 32.3 | 6.8 |
| 30 | 30.3 | 21.2 | 4.0 | 32.2 | 23.7 | 5.5 |
| 25 | 25.0 | 17.1 | 3.4 | 23.6 | 20.7 | 4.2 |
| 23 | 23.7 | 16.0 | 3.1 | 21.2 | 18.6 | 3.9 |
| 20 | 21.0 | 13.7 | 2.7 | 21.0 | 15.6 | 3.5 |
| 17 | 17.7 | 11.4 | 2.2 | 18.7 | 14.1 | 3.2 |
| 15 | 16.0 | 10.1 | 2.0 | 15.0 | 12.7 | 2.7 |
| 10 | 11.1 | 6.3 | 1.2 | 9.0 | 7.6 | 1.9 |

Henkel ink may reproduce resistors of which resistance values are closest to the designed values. Nonlinear resistor values are on average 3% (range: −1%-11%) larger than designed, and straight resistor values are 2% (range: −10%-13%) larger than designed. The smaller deviations from the target value in the case of nonlinear resistors would favor their usage over straight ones. Nevertheless, nonlinear resistors 42 and straight resistors 44 give rather similar values in the case of Henkel resistor paste. It should be noted that only Henkel ink was printed from a “fresh” jar because delivery of the PPG inks was delayed, such that “old” material had to be used. This may have had some effect on the results and they should be confirmed in the next sheet-to-sheet printing tests in which “fresh” material will be used. It is also possible that higher deviations in resistance values of straight waveguides is due to stretching of the TPU substrate 70. Further, nonlinear resistors 42 withstand stretching inherently better than straight ones and it is possible that this phenomenon is seen with PPG inks. In addition, in this test layout, the straight resistors 44 were printed along the printing direction, and the nonlinear resistors 42 were printed along a transverse direction, although in previous printing tests the orientation did not have a significant effect on the deviation of the resistance value.

Smallest resistor sizes behave similarly despite their shape. The nonlinear resistors 42 seem to give slightly more accurate resistor values when the designed values are between 40 kΩ and 75 kΩ, whereas straight resistors 44 give more accurate resistance values when the designed value is above 100 kΩ. PPG XRMC-78305 resistor values are not at the target values and PPG XCMC resistor values are on average 21-32% smaller than desired. The effect of the resistor ink and shape on the sample-to-sample deviation of the resistance is presented in Table 11.

TABLE 11
Effect of the ink and the resistor shape on the sample-
to-sample deviation of the printed resistors.

| De- | | | | | | |
|---|---|---|---|---|---|---|
| signed | Resistance deviation - meander | | | Resistance deviation - straight | | |
| resis- | (%) | | | (%) | | |
| tance | | XCMC- | XRMC- | | XCMC- | XRMC- |
| (kΩ) | Henkel | 50 | 78305 | Henkel | 50 | 78305 |
| 200 | 1.4 | 0.7 | 3.6 | 2.1 | 4.0 | 1.2 |
| 100 | 1.0 | 1.2 | 7.5 | 1.3 | 3.8 | 7.7 |
| 75 | 0.9 | 1.8 | 3.5 | 1.4 | 6.5 | 6.8 |
| 50 | 0.5 | 1.2 | 4.2 | 1.4 | 10.6 | 12.6 |
| 40 | 2.2 | 1.7 | 2.2 | 6.9 | 13.1 | 8.3 |
| 30 | 3.3 | 2.1 | 2.5 | 1.0 | 8.7 | 14.3 |
| 25 | 2.8 | 2.4 | 3.3 | 1.8 | 10.1 | 10.2 |
| 23 | 3.8 | 2.2 | 2.2 | 1.4 | 15.0 | 13.2 |
| 20 | 5.4 | 1.5 | 3.0 | 2.5 | 16.2 | 10.9 |
| 17 | 3.0 | 2.4 | 4.7 | 1.5 | 15.9 | 10.6 |
| 15 | 2.0 | 3.8 | 2.6 | 4.1 | 9.7 | 20.0 |
| 10 | 3.4 | 5.2 | 6.1 | 1.6 | 10.9 | 17.1 |
| Average | 2.5 | 2.2 | 3.8 | 2.3 | 10.4 | 11.1 |

Henkel resistors seem to have the most even resistor values. The average deviations are 2.5% (maximum 5.4%) and 2.3% (maximum 6.9%) for nonlinear and straight resistors 42, 44, respectively. PPG inks, XCMC-50 and XRMC-78305, can reproduce nonlinear resistors 42 rather evenly, but straight resistor deviations are on average over 10%, in particular with smaller resistor sizes. For example, XCMC-50 resistors have the average deviation of 2.2% (maximum 5.2%) with the nonlinear design, but with the straight design the average deviation increases to 10.4% (maximum 16.2%). Thus, PPG inks have more print unevenness than the Henkel ink.

### Effect of the Resistor Shape

The effect of the resistor shape on the resistance value and its deviation in the case of the resistors of the fourth test pattern 128 (e.g., short 10 kΩ resistors) is presented in Table 12. Straight resistors give resistance values that are farthest from the target value of 10 kΩ and have the largest sample-to-sample variation. All the other resistor shapes are rather near to the target value and the sample-to-sample deviations are low.

TABLE 12
Effect of the shape of the resistors on the resistance values and their deviation
in the case of 10 kΩ resistors. The line width is 1 mm.

| | ROLL3 - Henkel | | | ROLL4 - XCMC-50 | | | ROLL5 - XRMC-78305 | | |
|---|---|---|---|---|---|---|---|---|---|
| Field 4 | | | Std | | | Std | | | Std |
| 10 kOhm | Average | Std | (%) | Average | Std | (%) | Average | Std | (%) |
| Meander | 9.39 | 0.17 | 1.8% | 6.19 | 0.16 | 2.5% | 1.21 | 0.03 | 2.7% |
| Straight | 11.01 | 1.45 | 13.1% | 6.96 | 0.36 | 5.1% | 1.58 | 0.11 | 6.9% |
| Sharp step | 9.77 | 0.08 | 0.9% | 5.99 | 0.11 | 1.9% | 1.31 | 0.08 | 6.1% |
| Soft step | 10.15 | 0.22 | 2.1% | 6.45 | 0.32 | 5.0% | 1.45 | 0.13 | 8.6% |

### Effect of the Line Width

Table 13 shows the effect of the resistor width and ink on the resistor value and its deviation in the case of small 10 kΩ resistors. Straight resistors have larger deviations with XCMC-50 and XRMC-78305 inks than with Henkel ink, as already seen in Section 0. The sample to-sample resistor value deviations of the Henkel ink are the largest at the line width of 0.5 mm. This indicates that wider but longer lines can be more homogeneously reproduced. The line width of 1.5 mm seems to give resistance values that are nearest to the target value in this run.

TABLE 13
Effect of the line width and resistor ink on the resistor
values. The line widths are 0.5, 1, 1.5, and 2 mm.

| Field 3 | ROLL3 - Henkel | | | ROLL4 - XCMC-50 | | | ROLL5 - XRMC-78305 | | |
|---|---|---|---|---|---|---|---|---|---|
| Resistor | | | Std | | | Std | | | Std |
| width | Average | Std | (%) | Average | Std | (%) | Average | Std | (%) |
| 0.5 mm | 11.16 | 0.65 | 5.8% | 7.87 | 0.86 | 11.0% | 2.07 | 0.28 | 13.5% |
| 1 mm | 8.91 | 0.20 | 2.3% | 6.11 | 0.21 | 3.4% | 1.57 | 021 | 13.5% |
| 1.5 mm | 10.05 | 0.14 | 1.4% | 7.21 | 0.32 | 4.4% | 1.66 | 0.19 | 11.5% |
| 2 mm | 11.43 | 0.19 | 1.6% | 8.63 | 0.78 | 9.0% | 1.86 | 0.28 | 15.0% |

Table 13 shows the effect of the resistor width and ink on the resistor value and its deviation in the case of small 10 kΩ resistors. Straight resistors 44 have larger deviations with XCMC-50 and XRMC-78305 inks than with Henkel ink. The sample-to-sample resistor value deviations of the Henkel ink are the largest at the line width of 0.5 mm. This indicates that wider but longer lines can be more homogeneously reproduced. The line width of 1.5 mm seems to give resistance values that are nearest to the target value in this run.

### Effect of the Line Length.

The effect of the line length on the resistor value is shown in Table 14. As the resistor length increases, the resistance value increases, as expected. The sample-to-sample deviation of the resistance seems to decrease when using longer resistors (≥4 mm).

TABLE 14
Effect of the resistor length and ink on the
resistor values. The line width is 1 mm.

| Field 7 | ROLL3 - Henkel | | | ROLL4 - XCMC-50 | | | ROLL5 - XRMC-78305 | | |
|---|---|---|---|---|---|---|---|---|---|
| Resistor | | | Std | | | Std | | | Std |
| length | Average | Std | (%) | Average | Std | (%) | Average | Std | (%) |
| 1 mm | 1.49 | 0.09 | 6.0% | 1.45 | 0.47 | 32.3% | 0.33 | 0.07 | 21.3% |
| 2 mm | 2.70 | 0.13 | 4.7% | 1.84 | 0.24 | 13.3% | 0.57 | 0.16 | 28.1% |
| 4 mm | 4.93 | 0.10 | 1.9% | 3.33 | 0.32 | 9.7% | 0.92 | 0.14 | 14.8% |
| 6 mm | 6.98 | 0.22 | 3.2% | 5.20 | 0.40 | 7.6% | 1.30 | 0.20 | 15.4% |
| 8 mm | 9.11 | 0.12 | 1.3% | 7.35 | 0.93 | 12.7% | 1.80 | 0.36 | 19.8% |

### Effect of the Widening of the Resistor Line

Table 15 shows the effect of the resistor line end widening on the resistance value and its deviation. XCMC-50 and XRMC-78305 inks have very large deviations due to difficulties in reproducing straight resistors evenly. With the Henkel resistor, the resistance deviation does not seem to be affected by the width of the resistor line end. The end widths of 2 mm and 3 mm seem to produce resistors having the resistance values nearest to the target value of 10 kΩ.

TABLE 15
Effect of the widening of the resistor line ends and ink on
the resistor values and their deviations. The resistor length
and its width in the middle (1 mm) are kept constant.

| Field 4 | ROLL3 - Henkel | | | ROLL4 - XCMC-50 | | | ROLL5 - XRMC-78305 | | |
|---|---|---|---|---|---|---|---|---|---|
| Widening | | | Std | | | Std | | | Std |
| resistor | Average | Std | (%) | Average | Std | (%) | Average | Std | (%) |
| 1 mm | 11.01 | 0.40 | 3.6% | 7.07 | 0.53 | 7.5% | 1.85 | 0.29 | 15.9% |
| 2 mm | 10.33 | 0.31 | 3.0% | 8.03 | 1.92 | 23.9% | 1.95 | 0.25 | 12.6% |
| 3 mm | 9.83 | 0.23 | 2.4% | 8.90 | 1.36 | 15.3% | 1.80 | 0.27 | 14.8% |
| 4 mm | 9.11 | 0.29 | 3.2% | 9.22 | 1.49 | 16.1% | 1.96 | 0.32 | 16.5% |

Technical effects include systems and methods for printing biopotential sensors, such that the biopotential sensors may sufficiently withstand a pulse signal, for example during defibrillation. In some embodiments, the biopotential sensors may be printed using roll-to-roll methods, such that various layers may be printed onto a flexible or conformable substrate. In some embodiments, the substrate may be of any suitable material that fold and/or stretch, such as Thermoplastic Polyurethane. In some embodiments, a variety of electrodes, a variety of resistors, and a variety of signal traces (e.g., wiring) may be printed onto the substrate. For example, the electrodes may be printed with an ink having a composition that includes silver or silver chloride solution. The signal traces may be printed with an ink having silver. Further, the printed resistors may be printed in various sizes (e.g., length, width, shape, etc.) with various inks, as described in detail below. In some embodiments, the resistors may be coated with a material more thermally conductive than that of the signal traces. Further, in some embodiments, the final printed biopotential sensor may be protected from defibrillation pulses, require less resources to manufacture compared to conventional biopotential sensors, and may be foldable, such that it may accommodate various users of various body types.

This written description uses examples, including the best mode, and also to enable any person skilled in the art to practice the disclosed embodiments, including making and using any devices or systems and performing any incorporated methods. The patentable scope is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A biopotential sensor configured to withstand a defibrillation pulse, the biopotential sensor comprising:
a conformable polymer substrate;
a plurality of electrodes printed on the polymer substrate;
a signal trace printed on the polymer substrate;
one or more resistors printed on the polymer substrate and in electrical communication with an electrode of the plurality of electrodes via the signal trace, and wherein one or both of the one or more resistors and the polymer substrate are configured to withstand a defibrillation pulse; and
a coating layer applied to the one of both of the one or more resistors, wherein the coating is more thermally conductive than the polymer substrate.

2. The biopotential sensor of claim 1, wherein the polymer substrate is a thermoplastic polyurethane substrate.

3. The biopotential sensor of claim 1, wherein the one or more resistors comprise two resistors connected in series with the signal trace.

4. The biopotential sensor of claim 1, wherein the coating layer is configured to dissipate heat in response to the defibrillation pulse.

5. The biopotential sensor of claim 4, wherein the coating layer has a larger heat capacity than the polymer substrate.

6. The biopotential sensor of claim 1, wherein at least one of the one or more printed resistors are shaped in straight lines.

7. The biopotential sensor of claim 1, wherein at least one of the one or more printed resistors are nonlinear in a plane.

8. The biopotential sensor of claim 1, wherein at least one of the one or more printed resistors comprise one or more turns, curves, or angles.

9. The biopotential sensor of claim 8, wherein the one or more turns, curves, or angles, are along more than two directions, and the at least one of the one or more printed resistors has a three-dimensional arrangement based on the one or more turns, curves, or angles along the more than two directions.

10. The biopotential sensor of claim 1, wherein a resistor of the one or more resistors is coupled to a first contact pad of the signal trace at a first end and a second contact pad of a second signal trace at a second end, the second signal trace being configured to electrically couple to a patient monitor.

11. The biopotential sensor of claim 1, wherein the defibrillation pulse has a peak amplitude of 5 kV.

12. A method of manufacturing a biopotential sensor, the method comprising:
printing a plurality of electrodes onto a polymer substrate, wherein the polymer substrate is conformable;
printing one or more resistors onto the polymer substrate;
printing a signal trace onto the polymer substrate, such that the signal trace electrically couples the electrode and the one or more resistors; and
applying a coating material more thermally conductive than the polymer substrate to the one or more resistors, such that the coating material dissipates heat of the one or more resistors during operation of the biopotential sensor.

13. The method of claim 12, wherein the coating material is an insulator layer.

14. The method of claim 12, wherein the coating material is printed onto the one or more resistors.

15. The method of claim 12, wherein the printing of the plurality of electrodes, the one or more resistors, and the signal trace is performed via a rotary screen printer.

16. The method of claim 12, wherein the printing of the plurality of electrodes, the one or more resistors, and the signal trace is performed via separate printing steps.

17. The method of claim 12, wherein printing the one or more resistors comprises printing at least one resistor having a width between 0.5 millimeter and 4 millimeters.

18. The method of claim 12, further comprising separating adjacent resistors of the one or more resistors with one or more additional layers of coating.

19. The method of claim 12, wherein printing the one or more resistors comprises printing onto the coating material, the coating material being printed on the polymer substrate.

20. The method of claim 12, comprising pre-heating the polymer substrate.

21. A system comprising:
a biopotential sensor, comprising:
a plurality of printed electrodes, wherein the plurality of printed electrodes are configured to contact a user to detect a bioparameter associated with the user;
one or more printed resistors in electrical communication with an electrode of the plurality of electrodes via a printed signal trace, wherein the plurality of printed electrodes, the one or more printed resistors, and the printed signal trace are printed on a polymer substrate; and
a coating applied to the one or more resistors, wherein the coating is more thermally conductive than the polymer substrate.

22. The system of claim 21, wherein the biopotential sensor comprises an additional coating positioned between two printed resistors of the plurality of resistors.
